SEQUENCE LISTING (50 sequences)

SEQ ID NO: 1 - DNA, 143 bp, Bos taurus
  misc_feature (72)..(72): n is a, c, g, or t
ggctgcggggagcttggagaggggacgcccccgggcaggggtgcggtgtcgcgcgcggaaaaaccctgcganccggggcgcaggcggtaggaggagtcccgggcagataggctccaggagagggattccgggaaacggcggcg

SEQ ID NO: 2 - DNA, 143 bp, Bos taurus
  misc_feature (72)..(72): n is a, c, g, or t
tagcttgcaagaacacacaactgacaatagatttttctggtgagtttgtattacgttttcccttgtgatcangtgcagtgcagccactggtgagctggaacatggaaggcataacagttatttctagagtgggcaacggtggg

SEQ ID NO: 3 - DNA, 143 bp, Bos taurus
  misc_feature (72)..(72): n is a, c, g, or t
attggcagacagattcttcaccactggaccaccygggaagcccaggggactatttttaattactttttctanggtacgagagcatttgcactaattctacttgtatacatataaactctttatacatgcccacatgcttaact

SEQ ID NO: 4 - DNA, 143 bp, Bos taurus
  misc_feature (72)..(72): n is a, c, g, or t
gctgggagactgactcctgaaagccaaagcaaaacctcagaagtactgaaaggtagacccgtagtcaaaacngcagtgccattctgtatccttcccttccacttcttttcttttttctttcgcaatctctcactttatacaac

SEQ ID NO: 5 - DNA, 143 bp, Bos taurus
  misc_feature (72)..(72): n is a, c, g, or t
cttattgctttttgtttgttctgattattcagtgggctctctctttcccaggtgaaaacaaacggagtctcngccgtcagactggactcgccattaaagagtgacccatttgaagacttgtcattgaacctgcttgctgtatc

SEQ ID NO: 6 - DNA, 143 bp, Bos taurus
  misc_feature (72)..(72): n is a, c, g, or t
gtagtggagggaggcccgggtccccgcgcaccgtagcttccccagcctcagggtctgcccttgagccgtggnccccaacccccgccctcagatcgcgtttctctgttgctcctggcagcaggcacttctgagagcgggtactg

SEQ ID NO: 7 - DNA, 143 bp, Bos taurus
  misc_feature (72)..(72): n is a, c, g, or t
ctgagagcgggtactgtttccccctagtcccctccctctccccgctcccagatcgtgtttctctgttgctcntggcaacagccacttccgggagccgagccgtttcccccagttcccctgcccagtcccagcaccgcgaacgc

SEQ ID NO: 8 - DNA, 143 bp, Bos taurus
  misc_feature (72)..(72): n is a, c, g, or t
tttttagctccaaatgaaaaaatgaagcaagttataaagaagactatagaagaagccaaagcaataatatcnaaggttagctttttaaatgatattctaaaacttgtcttttgaaatacaaattccagttaatggcttccaga

SEQ ID NO: 9 - DNA, 143 bp, Bos taurus
  misc_feature (72)..(72): n is a, c, g, or t
ctgcctgccaggggctctctgaccctgcgagccttctgcggggggcgggggggagcaatgagtggaccaggntagasmcccagggattacgccaaggaccaaactgaggaggaggagagacgtcagggtgaagaggccagggc

SEQ ID NO: 10 - DNA, 143 bp, Bos taurus
  misc_feature (72)..(72): n is a, c, g, or t
ggatgctctaccctctagtgtcagctcacaaaccacaaacacatgcaaagcacatggtctgtcttgttgagnttcagtcactaagtcatgtccagctccctgcaaccccaygaactgcagcatgtcaggcttccctgtccttc

SEQ ID NO: 11 - DNA, 143 bp, Bos taurus
  misc_feature (72)..(72): n is a, c, g, or t
ttaccttcagacagtyccaaacttgggtggtaacctatagactgcttcaaggattgcaaccgcttcactaangaaacaagctctaagtgcctgtaatctgtgagaacaatctaatggttacaccgcaacggtacctaaagcaa

SEQ ID NO: 12 - DNA, 143 bp, Bos taurus
  misc_feature (72)..(72): n is a, c, g, or t
taaagcaatatttgttcagttacagtgacgtcacactgtagaaaggcttgttggctttctgttctttgctcnacaatattctcagcctgcyggtggctgaattcagattgctttccatagcttaaatcagcaaagacagaact

SEQ ID NO: 13 - DNA, 143 bp, Bos taurus
  misc_feature (72)..(72): n is a, c, g, or t
ttacagtgacgtcacactgtagaaaggcttgttggctttctgttctttgctcmacaatattctcagcctgcnggtggctgaattcagattgctttccatagcttaaatcagcaaagacagaacttttctagcagycaagaaag

SEQ ID NO: 14 - DNA, 143 bp, Bos taurus
  misc_feature (72)..(72): n is a, c, g, or t
cagtcataaattgagcctggagagagtattgtttgcttttctaacatgtttttcttctttccagtgcacatnagcctcgtgtttggtatttcgtatgttgcgcctggtaagagattytcttgacttaacaaaattttgtctaa

SEQ ID NO: 15 - DNA, 501 bp, Bos Taurus
  misc_feature (251)..(251): n is a, c, g, or t
cgggattccgacccaggcagcctggctccggagtctgttctcagacacccagccccatgctgaagggggccggattgctgggatcgctcagtgcccatcagcttgttactctagcctgctctcagaaagccctcataccagcccagttctaaaattctcaagtgaaagacagagtgttttcctggctgaggaaggagaccgtgtggcgaagagggaagagctgtgaggagtccaggaagtgacgtgtgccnactgtccagcacagacgtctgcgaggacgttgcagcatagttttggaagttctctggatcctaagcaaaaccacaacgaccagataaagtttttcctccatgggaaacatttttaactaaacaaaggcaacccccacatacagttcaaatgaaatccgcccacatggtgttggcatccgtgcaggaggaagcagtgaaagaacccaagcagtcagcagtgtcctgtgcaaaggcagcagcgcagtctgag

SEQ ID NO: 16 - DNA, 101 bp, Bos Taurus
  misc_feature (51)..(51): n is a, c, g, or t
ctcacctgcagcgccttctccttctacccacctgagctgaagctgcgcttnctgcggaacgggctggccattggctctggtgagatagacatgggccccaa

SEQ ID NO: 17 - DNA, 501 bp, Bos Taurus
  misc_feature (251)..(251): n is a, c, g, or t
tctctcatggtgaggattcttccggatgtttgatgagtctgggtttttttgtgctcatctcttttggggttgcactgggtcttctttgcaacatgcaggcttagttgccctgtagcatgtgagatcttagttctttgaccaaggattgaacccacatcctctgcattggaaggcaaattcttaaccactggaccaccagggaagtcccttgtgctcatctttatttggttaggaaactctcctccgtcagntgctggtgaatgcagctcttcctataaccagccccaaggaaaggggcgtggtctacttctgatgtggagctctggttttgttttgtggttgtggggagactgccctgcttctccctagggctgaagtctcttggggcatgacctcggctccctggacccaccctttagcccaggcacagtctcctgctattggctgcttgggcgaagctgggtaaagatggggatggccagcagggactcaatcccaggc

SEQ ID NO: 18 - DNA, 101 bp, Bos Taurus
  misc_feature (51)..(51): n is a, c, g, or t
gcttggggcttgtgctctcctagggcctggagaagccacttggagctccgnagccttcagactgcttcctcagccacctggcagtgaagcaggacagcctg

SEQ ID NO: 19 - DNA, 101 bp, Bos Taurus
  misc_feature (51)..(51): n is a, c, g, or t
ggcagtggctgcagctctgactccagcaacaccaagtcggctgcgggaggnggggccttttcacaaacgttgccttttataagtgtcttcatggatcgcta

SEQ ID NO: 20 - DNA, 101 bp, Bos Taurus
  misc_feature (51)..(51): n is a, c, g, or t
gagagcgatcagcaggagtctctgcacaaactcttgacatccgggggcctnagcgaggatttccgttcccattatgctcaactccagtccaacatcattga

SEQ ID NO: 21 - DNA, 501 bp, Bos Taurus
  misc_feature (251)..(251): n is a, c, g, or t
ctttggccatattcttttggtagaagaaggtcacatatcccacccacactcaaggggaggggattatacaggggtaagaatacaagaagctggggagcactggggatcacctgaccatttgggcatcatgcaagctgattggatcatcctcattccctttgacagtgaatagtttagtcatgagcatgtgacctactcctggctgatgggaagtaagaggataccttatagggtatgtggcagagactganggcacttgcagtatgcacattcccctcttcttccagggcacagaattagacggtatttcctagcctttttacatcttagtagggttgtataactaatggaatttcactaatggaatatgaatagaagtgaggtatatcactttctgcaccttctccttaccatctcttctttcactcacagatgctgaagatccagaagatattccaaggccttgaaggatgctggagccattagctggaatgagtctgg

SEQ ID NO: 22 - DNA, 501 bp, Bos Taurus
  misc_feature (251)..(251): n is a, c, g, or t
ccctccagagcagggaccggggaggtatacatggattgatcaggtactcagtacagggctatgggcttctgcttagctataaccagaacctcctgccaaaggtgggagcagttctttgagacccaagttatggaggtttctgagacccccctccctcaaaaacctggcttctggctgcttcaagggttgttgttgtttagctgttgtgtcctacctctttgagatcccaaggactgtaacccaccaggctnctctgtgcatgggggtttttcaggcaagaatactggagtgggttgccatttccttctccaggggatcttctcagatcaaggattgaacccgcatttcctgcattggtaggcagatactttaccactgagccacctggaaagccctgcttctagggtactgccttattcttattcactctcttcccctaaagcccattccagtttctatagagacaagggttcctaggttcatagagttctgggttgtctg

SEQ ID NO: 23 - DNA, 501 bp, Bos Taurus
  misc_feature (251)..(251): n is a, c, g, or t
actgcctatttacactgtgtgggcctttgtaatgactgctatcattacagtgtgctagacgatactctattcttcacatgtttattctttagtcttaaaacaattcaaaagcataggtacagtacaaattaatcatttgaaacctctactataacagacttgagaggccagaaagagaagctttcacaccctatgataataacagagcacaaaaggaagaagaaagacttcctcttctcgctgggcaacanctcagccaatgagaaactgtcagaagtcagccaatgaaaaaccactgtacttagaactctcagttcctcaaatagaacctttgtttatcatagccctcctttcttttccctattatatctactctacttgttgacctggattttcacatggcttatttttgcagaatctgaatcacacttctttgttatcccaaataaacctatctttcactgtagaaataactggcaatctatttatgtaaaatgagca

SEQ ID NO: 24 - DNA, 501 bp, Bos Taurus
  misc_feature (251)..(251): n is a, c, g, or t
catcctactgtcccataaaattaatctttaaaatgtaaaaggacgagggtttttgttttgtttttcttagtgaataaagacaagtgcaaggaaaaaagttggatttgtgtcaattactaatcgcaacttattgatcattgacatctttaaatgtctgatcggatgccttagatttgtattagctgaaaaaacattagtgacgactgatgaagttgttttgcatttccatgcctggctcatagtacagactnaaaaacacaggcttcactcaagggacacagaatgatgaaaacctgaagggtgaaataagtctagctgaaaaaagcgttattcaagataagtagggagtgtctttgatgtgaaagatgcaggagggaagagaaaagtggtaaaaaaggttagttaatgaacacaccatgttcagtgccctgtgggagactgctcaaacataacttgcacaaaaatgaatccatctagtgaaggacaccaccatctctccag

SEQ ID NO: 25 - DNA, 501 bp, Bos Taurus
  misc_feature (251)..(251): n is a, c, g, or t
gaggatcagctgaggaaaatggcaaagttgatgttttctgcaactgtctgtcattgttatttcaaaagaaagaataaatttgggtctctaaattgtcacagttttattttctaatacagtaaatatggatggataacacccacaagataaaagttctttgggacccttaataatttttaagtgtgtaaaagtgtcctgaggcccaatggcttaagaactgctgacacagaatttatgaggagaccaatganaaagggcagagcctggggagctgggcagccctggacggactggacaaagcgaggggtggtgtccacgttccccgggcaggctgggctgcaagcgtccctgggcagagactcaaactcggccaccaccgggaaaggtgcaggctccacttccgcctcagacagacagagcaggacgcagggggaggcccagtgccgcctgtgacgtgaggggaggtctgggatgtccccagagggcacccccagggcgtgg

SEQ ID NO: 26 - DNA, 501 bp, Bos Taurus
  misc_feature (251)..(251): n is a, c, g, or t
taactcaaatatctggaatttttacagttgtcctttgttttgtctctgaaagggactgagggtatctattgcatcggtgaaacttagggagaaagggattggagattaaatctggaggaaataacatcttaaatacatgctttttataaggttcataataaatattatgaatgaagtaaagacacatggaagggaagcccattgtgagaaaacattcatagtatatgtgaaataacactgcaagccccaanggctatttgctataacacaattcttatagttagttccttgtgaatgattcattcggtcagttcagttcagtcgctcagttgtctccgactctttgcgaccccatgaaccgcagcacagcagacctcactgtccatcaccaactcccggagtccacccaaatccatgtccattgagtcggtgatgccatccaaccatctcatcctctgtcgtccccttctcttcctgccttcaatctttcccagcatcagg

SEQ ID NO: 27 - DNA, 501 bp, Bos Taurus
  misc_feature (251)..(251): n is a, c, g, or t
ccctgtgggttcgcctctgcgggattttcatctgaatagaatcctgcaacacgagcaacgctgtgactgacttcttttcacccagaaccttgttttcaaggttcatccgggccaacgatgcatcggtatttcatttctttggattgctaaataatactccactgtatggacataccgtactttatttatccaccagtggatggatattgggcatttgtccactttttgtctactgtgaattgaccgatgcngctatgaacattcatgtacaagtttttgtgtagacatatgttttcatttctgttggttatatacaaaggattggaatagttgagtcatatgctaactctgttaacttttgtggaagtgccagagaagctgttcattttctagacattctgatgatctggagttaaaaaaagtctaatctgactttctacttctatgaaactgctgtttggtgatgagattattttaagtgagggatagaaagtcaaaaga

SEQ ID NO: 28 - DNA, 501 bp, Bos Taurus
  misc_feature (251)..(251): n is a, c, g, or t
tgggtcaccagaacaccctcaggccctctgttaactcccagggttgtttcctctgactcccccaacaccacagccctcccaccgtggacactgtgtattctgtgcctcagacccacactagcacctcccttccctccattcccagaacttcttagttcatttttcttccagtgatttgttactttcatacttatttccccaatatccttgtgtctatttttaggctttctattttatcccattgatccatntcctcaaccactggctccacattactatttgttgaccttgcagtcagagcagagcccggtgatcaccctcccgcagccctgtcaatgcagacgctgcacactgcaggcctcccacccacaccaccgccctcaggccctctgctcactcccacggttgtctcctctgactcccccaacaccacagccctcccaccatggacactgtattctgtgcctcagcccacactagtacttccattccctctcaggc

SEQ ID NO: 29 - DNA, 501 bp, Bos Taurus
  misc_feature (251)..(251): n is a, c, g, or t
tgtgctgctaagagttctctttagtgtccgtaatgggcttttataggctattttgtggacatctcccacttgggtggaaggaacacctctttatcataattcctaaaatgtttcacaattggaacatatcaaggtaatggttccatcttcccatctccatggaataaccatgactttttctttctctaaaccactgtgatattgtcagccccacttagcgtagcagttaatcagcctcctcctttcattgntcatctgtttcacttttgagtaactttatttcccagaccaaacataagctgttcaaggacagagtgtttttcatctcactttgattttccccggcccacggctgcaaccgagcagagtcaggttgtcaccaaatacctggtaaatgaatgggatccagaagggaaagtaaaactaaatgtcaggagtatgggcgcgtggtccaggattcatggaaattagaaaagctttatgttacaaactaaaacaatt

SEQ ID NO: 30 - DNA, 501 bp, Bos Taurus
  misc_feature (251)..(251): n is a, c, g, or t
gtgtcacctaaacaatgatatcaaacttggggatttctgccaatctgatgggtgaaaatggaatctcggtgtatttttaatctctggtagacttcgcatgaataaggttgaggatttcttcaaaagcttaaaagtcatttacgtttcttatttgctaatggcttctggttttgcgtcatataaaggtctttcaaactccaaagttaatcagaaacccttctatgttttcttctgccgtttttatggtttcntaatgtatccactggttcatggaactggaagcttttcttgtcattttctcagcggtgagcacctagctggatcctgaaaggcttggggtctgggtagagacttgagctgccagtggagccctctaggtgctgggctccaatccccttcagctctgccagaagtctcagccagcagaccctccaccttcctccctccagcgcgcctgtgtcctgggctggcacagggggcccagagttgaggcggtccctg

SEQ ID NO: 31 - DNA, 501 bp, Bos Taurus
  misc_feature (251)..(251): n is a, c, g, or t
acaattttcatcattttctgtctcttgtacctccttccatgtgagagaagcctttgtattggcagagaactgagggaagaacggcagtgtttagttgaggcagatttcttcatctcagattatattttgggagagacaggccatgctgcagaacttggagaaaagacgatgagggtgacaggccctctttctattttcatcttcccctttcttctttgttatgtttctaggaattataaataatgccttcngtcacagagctttcctactaatggtcactttcccccaagcctctttcagagttgacaggccactgcactccccagatggttagggagacttggagattgaggtcacagctgtcaactccagagcactgtggctgacacatcatacttactgcaaacagagggagcttgcatgagaaattagagcagttgggacttacccagtggccagtggttaagactccaccttccagtgcagggttcgatctctgtg

SEQ ID NO: 32 - DNA, 501 bp, Bos Taurus
  misc_feature (251)..(251): n is a, c, g, or t
atctgacttgcatggtctctgcctgtccattcagtggaggccacatgcctcatttgtgtcctttgacaccatctggttcccactccctcaccaacctgctttttcagggccatcattttgggttgaggaagtgtacgctttagctctgtgatgacaagatacatatcaactgctgcttttcctgcctgacaacttcctaggaagtccagagactaactttgggacaagaaggtttctctcctatcctttcngtactactaaagcctttgactgtatggatcacaataaactgtggaaaattctgaaagagatgagaataccagaccacctgacctgcctcgtgagaaacctatatgcaggtcaggaagcaacagttagaactggacatggaacaacagactggttccaaataggaaaaggagttcgtcaaggctgtatattgttaccctgcttatttaacttatatgcagagtacatcatgagaaacactgggctggaaga

SEQ ID NO: 33 - DNA, 501 bp, Bos Taurus
  misc_feature (251)..(251): n is a, c, g, or t
gaggggctggggggcatgctttaaaggtctcactagcatctcatttgctctttggagagttgggtttggcagcagagaggggagggcctctttggcttccaggaggaaatgttctcctcctctctttcctgttaaccttagtccagagtatggaggccaactcctccttgcgagggaagctttgatctgactaccaggcttggaccggcttgacctgagataatgttggataatgtaacaaacaagctacntgtaatatcagctggtcaatgagtagcctccccaaccccaaggcaaagcttgacattgctgttttaaaataataatctataatcatttatgtatatcttaagctcaaggcccaatagttcaaaaataaaaggtgagtacagagattcttctctgtacttctattttaatttgggggtgagggtcatttgctttcaaacctaggtgatgttctatatctattagggcccccaactgaataaagtattagac

SEQ ID NO: 34 - DNA, 501 bp, Bos Taurus
  misc_feature (251)..(251): n is a, c, g, or t
ggccagggtccctcgttagcaactggaggtttaaagcaggtaacttgacacatctcaaattagccttccttccttgtaaattgacgggcgaaatcgctccctcttgaaagtgcacggagaattaaaacacttagcatggtgtgttctaaagacagggaggtaccttttctaatgcacagaagccctgagtgtggctgtggattcagcctgactatgagtcctgatggctctaagtatggctgtaattgatngcttaaagtgatggtggtaatgatccttatcttttgaatgtttgtgatcagaactaacgatgagtaagtacaaacagtgatcttagacgagcaagtccctacccctggcttcagtccagtctgaacaccccaaggtcggcacagaaatgggctgcccagtgacccctctgctaagtaaggctgaccgcaccttcagtcacctgtccatctcttcagcaaacatctatctgctacatgcctatgctggacg

SEQ ID NO: 35 - DNA, 501 bp, Bos Taurus
  misc_feature (251)..(251): n is a, c, g, or t
gggcgatcaggaaggacactgtgagggcggccctggatggatgtgtctttgtccaggtttctgaggggtgggtaaagtgaaaacaagccccctttgggtctctgcgacctgagggcccctctgttactctcactttccctctttcagcgttttcaaatctcagatgttttgttcagacgacgtgcctctggctctcctcatcaggaagttggaaatttcaggaaggaaatctggatcactgcatatcacanggaatggtgtatttttggatttcagaaagaatggggtctctggcaaggagtcactgagatcatgccttgcgaacaggaaggatgtatcgggtcacttacattgagccactggtgaacgtttaacgttagctgggtcttggggggaaaagccccaatttgtatgtagcgtttccctattcctgtggtgtaaatactcccaccagggcacactgcagggtaccaaatagctgtccttatacacagagctggg

SEQ ID NO: 36 - DNA, 101 bp, Bos Taurus
  misc_feature (51)..(51): n is a, c, g, or t
tctgcacttcactcggtaatattagcaaatctccaaatgttagccacattngtttgtttcccttgtatgttgtttattcatgatacttcaatgctgtaact

SEQ ID NO: 37 - DNA, 501 bp, Bos Taurus
  misc_feature (251)..(251): n is a, c, g, or t
tttttttttttttggaaagtgcttaaattaattgaacttgtccaatcaacttttttttttttaagtctccttcatagtaatatccattgggacttagaagttacacaaattgagtaactgatctgtaaagaaatactgagaatatagtgaatggtagcctttatcaggagagggcacatactagaatctttcttgtactcttttcttgagaagcgttaacgaaactcttggtggaacatacttgataacanatattcatctaacatcagggaaatgagaaggttagacatttggacctaggatactcaagacaacagtatatcagagctgtcgtgtttagttcactcaaacagaattttaagttactctaaaatttctttcaattagtgaaaccttagctctcatgaaaggtactagattgacaacccctgaggacactctactttgtcaccagcacagcagcagatgccacctgccagcagtccaggtatcacaagcccg

SEQ ID NO: 38 - DNA, 501 bp, Bos Taurus
  misc_feature (251)..(251): n is a, c, g, or t
taatagtcactcaatacaggctctcttcaagaatctaaatgcggaaagctattcattcgtaggaagctggtcgtgcgtgttttgtggaattcagacgaatgtcctggggtcaggatttttagaaagtcataattcggtctttagatttcatatgactgttatgctttaatctggaaagagcagaaatcagtcagatgaaatttgagactgtctactcttggtccgtaatggtaactcagatggaacgattnttttgaatcatgaaaagggaatttcttggtgtcagcttataagatcctgaagtcagtcagaactcacagatacaagcctatttaaacgaggcctccctgctgcccctttcttggaaaatgcacctgcacttggtgtcatattattgtctatatttgtgtgtgtgtcgaagggcttggcagtttcatatgagccaaatgagctgaaatctttgtttaaaaaaattctttctgtgtccagggtctagaaccc

SEQ ID NO: 39 - DNA, 501 bp, Bos Taurus
  misc_feature (251)..(251): n is a, c, g, or t
atgataagttatatggttttcctctggtcactgaaatactggcaggaagcagaatttcgaggaaaggtcagtagttaagttttgaaactgttcagtttgatttgtctacaggagatgtggaaagaagcttgagtagacaattagatacaagtcttttctttatgctagctgtattgagatataatataattgatatatagaactgtataattttaaggcttagagcataatgttgtggcttatgtatattntgaaatgatgactacagtaagtttgattacatccatgtagctctcatacagctaaaaagagaaaaacttttctattttcctttgtgataaaaactcttaggatgtactctcatatagctgtgtaaattacagtcatgtggtacatcatatccccagtttttatttatcttatccctggaagactgtacctattgaaaagctggtttttaacatattcttgtgaaaaatgctgcacatgcttcctctcagt

SEQ ID NO: 40 - DNA, 501 bp, Bos Taurus
  misc_feature (251)..(251): n is a, c, g, or t
tagttatacttcattaatttttaaagaagattttatatccttctactctgacaagttatgaatcaattttaaaagctctgtgtttatcttagaatcctcctacacattaagaaaaatcttcatcattgaatttgatttagagaatgcatatgaagaaaaagaaaacacattgaaaaatatttctctttttctgactatgttttatatgccccagctcagtatatattaattttgaaagccaggaaaaaaantggcagagattttaaataaataaatttggctgaaacattacagagcaaagcaaaagcataatgtacctgggataacagttatagccattcctcaaatgagtggcatcatctgcatatctgaggttgttgatatttctcatggcagtgttgattccaactcaattaaaagacccttgttcctttgaagaaaagctctgacaaacctagacagcatattaaaaaaatagagacatcactttgctgacaaagg

SEQ ID NO: 41 - DNA, 501 bp, Bos Taurus
  misc_feature (251)..(251): n is a, c, g, or t
gaaacaaactagtggttaccggtggaaagagggaagggggaggggcaaggtaagagaatgggattaaaaaatgcaaactactgtgtataaaatagataaattacaaggctatactgtacagcactgagaatcatggcattgtcatgcaatacattttaatggagtagaatctgtaaaaacactgaatcactaagctgtacacctgaaactaatataattttgtaaacagattcaatttttaaaataatcanttaagccatgagaaagaagaaacgggggaggcaagtggattactcaacacctgctgattcatctgtcttatttttatctcagcatacaattattaattcttttttctgccttttaaatttttaactaccaaggcactgctaaagtcagcatccgaagagttatattttaaactttaaacctgtcaaggagacaggattggacaggtaccagcattccagttcgggaatgagaccatctacttctacagcc

SEQ ID NO: 42 - DNA, 501 bp, Bos Taurus
  misc_feature (251)..(251): n is a, c, g, or t
ttttcccctcaattcccaaagaaacactgactcataatcacacacacacacacagtcatttggaaaaaattaatttaaaatctgtttttcttaagactttcaattatttcggccccatcactaagcattcagtttaaagaaaataaaaactctaggcaggcaaaatcatgaccaaagctatcattgtacagcatattgatggcatatctagaagtctgaaaccgagaagcaatatgaaaagttacttttgnatttagtgctcttttttttttagttttcaggtttgtagcctaagcattaagataaaaatgaccaggtggcagggccagctgattatactaaggcttccctgaagaactgcagctgcaagtaaattcttttctgaccagcctttattattggcagttatcacatagtatatgctgtccccctgtgctctcctgggacagtgggaaagacatggaaatgagaaggcagttggatgagggtggaagagatacc

SEQ ID NO: 43 - DNA, 501 bp, Bos Taurus
  misc_feature (251)..(251): n is a, c, g, or t
actactctgtgtatcaagattaagttattatggtttataataatacctctttttctacatcacttaataattttgtttctttcattgtaactccactgaatcctctgtgaaatgtactgtgggtgttcgtaaaatttagtacatggcaaagtctgggcttgaaattggctttcctcgtatccattagcaactctcttctagcatttcttatacagctttatgattttttaaactttctttagacataaacnatggaattctcaaatacagggacagtgtcttactcctatttatttctataaagtatagaccaaggtcccatcagtagttggtacttgatacatgtttggtgggtgaatggagagatgaaattttatctgatggtaatataaattttcatctctggagtcactaaagtttacataccattttgatttccagggtattttgagaacattttattgttgtagccagtctaacaggataggagatagtatgctt

SEQ ID NO: 44 - DNA, 501 bp, Bos Taurus
  misc_feature (251)..(251): n is a, c, g, or t
ccaacccagggatcaaacccaggtctccggtattgcaggcagattccttaccagctgagccacaagggaagtcaaagaatactggagagggtggcctatcccttctccagcggatcttcccgacccagggtctcctgcattgcaggcagattctttaccaactgaggtaccagggaagccccgatgtaaaactaaccttaatattcatactttgttgtgtcataggaaaaataaaactctaatattacttnctatttgtatgtttattccaggatagtgctaaaattgtaacttgaagctttttaaatttattactataatttaataagatttttaaaaacctcaagaaaatcaagggttttctgatacattgtgcagttaattgattaaataatgatgatgcataaactctctaaaatcttgaacttttaattcacttgacctatacagatcacctatacatagaactatgctgtattggggaatatcaatacatagcac

SEQ ID NO: 45 - DNA, 101 bp, Bost Taurus
  misc_feature (51)..(51): n is a, c, g, or t
caggttgcatgtggacactcagccacattaacaacttggagaaaaaaaatnaaaaaatggcaatgttataatgaattctcaggtgaacttttttttcagtt

SEQ ID NO: 46 - DNA, 501 bp, Bos Taurus
  misc_feature (251)..(251): n is a, c, g, or t
agagtcagacacaaaaggccacctactgtgtgattccttttatatgaaatgtacagaacagccaaagctatagagacagaaagcagagcagtgtttgacagggactggggagggtctacttaacactacaagatttccctttgagaatgtcctggaactagagagtggtcatagttgtacagtgttgtgactgtactaaatgtccctgaattgaacactttaaaatggttaattttatattatttgttacntgtaataaaaaacatacctgttaaggatttccaacagggaggcccctagagcttccctggtgatccagtggttaaaaatccacctgccaatgcaaagaacataagtttgatccctggtctgggaatagcccatgtgccacagctaccaaagcccatatgtcctaaagccaatgctctgcaataagaaaagccattgccaagagaagcctgcccactgcaacaaagagtaaacccaatttgccacaaccag

SEQ ID NO: 47 - DNA, 41 bp, Bos Taurus
tcttacacatcaggagatagytccgaggtggatttctacaa

SEQ ID NO: 48 - DNA, 41 bp, Bos Taurus; identical to SEQ ID NO: 47

SEQ ID NO: 49 - DNA, 41 bp, Bos Taurus; identical to SEQ ID NO: 47

SEQ ID NO: 50 - DNA, 41 bp, Bos Taurus
tcttacacatcaggagatggytccgaggtggatttctacaa

(19) United States
(12) Patent Application Publication
(10) Pub. No.: US 2010/0324356 A1
(43) Pub. Date: Dec. 23, 2010

(54) METHODS FOR IMPROVING GENETIC PROFILES OF DAIRY ANIMALS AND PRODUCTS

(75) Inventors: Edward J. Cargill, Cottleville, MO (US); Michael D. Grosz, Ellisville, MO (US); Nicholas J. Nissing, St. Charles, MO (US)

Correspondence Address: PFIZER INC;Mary J Hosley, 150 EAST 42ND STREET, MS: 150/02/E112, NEW YORK, NY, 10017-5612, US

(73) Assignee: PFIZER, INC.

(21) Appl. No.: 12/747,385

(22) PCT Filed: Dec. 15, 2008
(86) PCT No.: PCT/US08/86811

§ 371 Date: Sep. 9, 2010

Related U.S. Application Data
(60) Provisional application No. 61/014,904, filed on Dec. 19, 2007.

(51) Int. Cl. A61B 17/43; C40B 30/00; A01K 67/00

(52) U.S. Cl. 600/33; 506/7; 119/174

(57) ABSTRACT

The present invention provides methods for improving desirable dairy traits through the use of a genetic profile. Also provided are methods for determining an animal's genotype with respect to multiple markers used in the analysis of the genetic profile. In some embodiments, the genetic profile includes at least one marker associated with the polled trait. The invention also provides methods for selecting or allocating animals for predetermined uses, for picking potential parent animals for breeding, and for producing improved dairy products.

### PRIORITY CLAIM

This application claims the benefit of U.S. Provisional Application Ser. No. 61/014,904 filed Dec. 19, 2007, which is herein incorporated by reference in its entirety.

### INCORPORATION OF SEQUENCE LISTING

A sequence listing is contained in the file named “Polled_ProductSEQLIST_final.txt” which is 29,629 bytes (28.9 kilobytes) (measured in MS-Windows XP) and was created on Dec. 12, 2008 and is submitted herewith (in accordance with 37 C.F.R. §1. 1.821), and incorporated herein by reference.

### FIELD OF THE INVENTION

The invention relates to improved genetic profiles of dairy animals, products comprising improved genetic profiles, and methods of producing these products. More specifically, it relates to using genetic markers in methods for improving dairy cattle and dairy products, such as isolated semen, with respect to a variety of performance traits including, but not limited to such traits as, the polled/horned phenotype, productivity and fitness traits.

### INTRODUCTION TO THE INVENTION

The future viability and competitiveness of the dairy industry depends on continual improvement in a variety of traits including milk productivity (e.g. milk production, fat yield, protein yield, fat %, protein % and persistency of lactation), health (e.g. Somatic Cell Count, mastitis incidence), fertility (e.g. pregnancy rate, display of estrus, calving interval and non-return rates in bulls), calving ease (e.g. direct and maternal calving ease), longevity (e.g. productive life), and functional conformation (e.g. udder support, proper foot and leg shape, proper rump angle, etc.). Some characteristics, such as whether or not an animal has horns, can be important for the efficient operation of a farm as well as animal welfare.

Genomics offers the potential for greater improvement in productivity and fitness traits through the discovery of genes, or genetic markers linked to genes, that account for genetic variation and can be used for more direct and accurate selection. Close to 1000 markers with associations with productivity and fitness traits have been reported (see bovineqt1v2.tamu.edu/index.html for a searchable database of reported QTL), however, the resolution of QTL location is still quite low which makes it difficult to utilize these QTL in marker-assisted selection (MAS) on an industrial scale. Only a few QTL have been fully characterized with a strong putative or well-confirmed causal mutation: DGAT1 on chromosome 14 (Grisard et al., 2002: Winter et al, 2002; Kuhn et al., 2004) GHR on chromosome 20 (Blott et al., 2003), ABCG2 (Cohen-Zinder et al., 2005) or SPP1 on chromosome 6 (Schnabel et al., 2005). However, these discoveries are rare and only explain a small portion of the genetic variance for productivity traits and no genes controlling quantitative fitness traits have been fully characterized. Some genetic tests related to the horned/polled phenotype have been developed (see for example, US2007134701A1 and US2005053328A1). However, these tests have less than ideal predictive ability in dairy cows. A preferred method of testing is provided by U.S. provisional application Ser. No. 60/977,238, filed Oct. 3, 2007, herein incorporated by reference in its entirety.

These prior evaluations generally describe selection based on only one associated phenotype. A more successful strategy employs the use of multiple markers across of the bovine genome in which markers associated with multiple traits including horned/polled, productivity, and/or fitness are simultaneously used for selection.

Cattle herds used for milk production around the world originate predominantly from the Holstein or Holstein-Friesian breeds which are known for high levels of production. However, the high production levels in Holsteins have also been linked to greater calving difficulty and reduced levels of fertility. It is unclear whether these unfavorable correlations are due to pleiotropic gene effects or simply due to linked genes. If the latter is true, with marker knowledge, it may be possible to select for favorable recombinants that contain the favorable alleles from several linked genes that are normally at frequencies too low to allow much progress with traditional selection. Since Holstein germplasm has been sold and transported globally for several decades, the Holstein breed has effectively become one large global population held to relatively moderate inbreeding rates.

Multiple markers must be used in MAS in order to accurately describe the genetic profile of an animal without phenotypic records on relatives or the animal itself. In particular, selection based on numerous markers related to multiple different traits would be preferable. The application of such a multi-marker selection based on genetic profiles for homed/polled, productivity, and fitness traits is described herein.

The large number of resulting linked markers can be used in a variety of methods of marker selection or marker-assisted selection, including whole-genome selection (WGS) (Meuwissen et al., Genetics 2001) to improve the genetic merit of the population for these traits, create value in the dairy industry, and improve animal welfare.

### SUMMARY OF THE INVENTION

This section provides a non-exhaustive summary of the present invention.

Various embodiments of the invention provide methods for evaluating an animal's genetic profile at 12 or more positions in the animal's genome and methods of breeding animals using marker assisted selection (MAS). In various aspects of these embodiments the animal's genotype is evaluated at positions within a segment of DNA (an allele) that contains at least one SNP selected from the SNPs described in the Tables and Sequence Listing of the present application.

Other embodiments of the invention provide methods that comprise: a) analyzing the animal's genomic sequence at one or more polymorphisms (where the alleles analyzed each comprise at least one SNP) to determine the animal's genotype at each of those polymorphisms; b) analyzing the genotype determined for each polymorphisms to determine which allele of the SNP is present; c) analyzing the genetic profile of said animal, and d) allocating the animal for use based on its genotype at one or more of the polymorphisms analyzed.

Various aspects of embodiment of the invention provide methods for allocating animals for use based on a genetic profile using an animal's genotype, at one or more polymorphisms disclosed in the present application. Alternatively, the methods provide for not allocating an animal for a certain use because it has an undesirable genetic profile which is not associated with desirable phenotypes.

Other embodiments of the invention provide methods for selecting animals for use in breeding to produce progeny. Various aspects of these methods comprise: a) determining the genotype of at least one potential parent animal at one or more locus/loci, where at least one of the loci analyzed contains an allele of a SNP selected from the group of SNPs described in Table 1 and the Sequence Listing; b) analyzing the determined genotype at one or more positions for at least one animal to determine which of the SNP alleles is present; c) analyzing the genetic profile of said animal; and d) allocating at least one animal for use to produce progeny.

Other embodiments of the invention provide methods for producing offspring animals (progeny animals). Aspects of this embodiment of the invention provide methods that comprise: breeding an animal—where that animal has been selected for breeding by methods described herein—to produce offspring. The offspring may be produced by purely natural methods or through the use of any appropriate technical means, including but not limited to: artificial insemination; embryo transfer (ET), multiple ovulation embryo transfer (MOET), in vitro fertilization (IVF), or any combination thereof.

Other embodiments of the invention provide isolated semen comprising improved genetic content. Preferably, the isolated semen comprising improved genetic content further comprise genetic profiles as described herein. Various embodiments of the invention also comprise frozen isolated semen, and isolated semen with disproportionate sex determining characteristics, such as for example, greater than naturally occurring frequencies of X chromosomes.

Other embodiments of the invention include a method for allocating a bovine animal for use according to the animal's genetic profile, the method comprising: determining the animal's genotype at 12 or more loci, wherein each locus contains a single nucleotide polymorphism (SNP) having at least two allelic variants; and wherein at least 12 SNPs are selected from the SNPs described in Table 2 and the Sequence Listing; analyzing the determined genotype of the at least one evaluated animal; and allocating the animal or use based on it's determined genetic profile; wherein the animal is homozygous for the preferred allele for at least 12 SNPs selected from the SNPs described in Table 2 and the Sequence Listing.

Other embodiments of the invention also include a method for allocating a potential parent bovine animal for use according to the animal's genetic profile, the method comprising: a. determining the animal's genotype at 12 or more loci, wherein each locus contains a single nucleotide polymorphism (SNP) having at least two allelic variants; and wherein at least 12 SNPs are selected from the SNPs described in Table 2 and the Sequence Listing; b. analyzing the determined genotype of the at least one evaluated animal; and c. allocating at least one animal for breeding use based on it's genotype; wherein the animal is homozygous for the preferred allele for at least 12 SNPs selected from the SNPs described in Table 2 and the sequence listing.

Other embodiments of the invention also include a method of producing progeny from bovine animals comprising: a) identifying at least one potential parent animal that has been allocated for breeding in accordance with the method described herein; b) producing progeny from the allocated animal through a process selected from the group consisting of: (i) natural breeding; (ii) artificial insemination; (iii) in vitro fertilization; and c) collecting semen/spermatozoa or at least one ovum from the animal and contacting it, respectively, with ovum/ova or semen/spermatozoa from a second animal to produce a conceptus by any means. In preferred aspects of this embodiment of the invention, the progeny is polled.

Other embodiments of the invention also include a bovine product having a genetic profile wherein the genetic profile comprises single nucleotide polymorphisms (SNPs) and wherein the product comprises at least 12 SNPs selected from the SNPs described in Table 2 and the Sequence Listing and wherein the product is homozygous for the preferred allele of at least 12 of the SNPs described in Table 2.

Other embodiments of the invention also include a bovine animal having a genetic profile wherein the genetic profile comprises single nucleotide polymorphisms (SNPs) and wherein the animal comprises at least 12 SNPs selected from the SNPs described in Table 2 and the Sequence Listing and wherein the animal is homozygous for the preferred allele of at least 12 of the SNP described in Table 2. In preferred aspects of this embodiment of the invention, the bovine animal is polled.

Other embodiments of the invention also include a method of determining a genetic profile of a bovine product: a) collecting a sample of biological material containing DNA; b) determining the genotype of the biological material at 12 or more loci, wherein each locus contains a single nucleotide polymorphism (SNP) having at least two allelic variants; and wherein at least 12 SNPs are selected from the SNPs described in Table 2 and the Sequence Listing; and c) analyzing the determined genotype; wherein the biological material is homozygous for the preferred allele for at least 12 SNPs selected from the SNPs described in Table 2 and the Sequence Listing.

### Definitions

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “allelic association” preferably means: nonrandom deviation of f(AᵢBⱼ) from the product of f(Aᵢ) and f(Bⱼ), which is specifically defined by r²>0.2, where r² is measured from a reasonably large animal sample (e.g., >100) and defined as

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

where A₁ represents an allele at one locus, B₁ represents an allele at another locus; f(A₁B₁) denotes frequency of gametes having both A₁ and B₁, f(A₁) is the frequency of A₁, f(B₁) is the frequency of B₁ in a population.

As used herein the terms “allocating animals for use” and “allocation for use” preferably mean deciding how an animal will be used within a herd or that it will be removed from the herd to achieve desired herd management goals. For example, an animal might be allocated for use as a breeding animal or allocated for sale as a non-breeding animal (e.g. allocated to animals intended to be sold for meat). In certain aspects of the invention, animals may be allocated for use in sub-groups within the breeding programs that have very specific goals (e.g. horned/polled, productivity, or fitness). Accordingly, even within the group of animals allocated for breeding purposes, there may be more specific allocation for use to achieve more specific and/or specialized breeding goals.

As used herein, “semen with disproportionate sex determining characteristics” refers to semen that has been modified or otherwise processed to increase the statistical probability of producing offspring of a pre-determined gender when that semen is used to fertilize an oocyte.

As used herein, the term “bovine product” refers to products derived from, produced by, or comprising bovine cells, including but not limited to milk, cheese, butter, yoghurt, ice cream, meat, and leather; as well as biological material used in production of bovine products including for example, isolated semen, embryos, or other reproductive materials.

As used herein, the term “isolated semen” refers to biological material comprising a plurality of sperm/semen which is physically separated from the originating animal, typically as part of a process employing human and/or mechanical intervention. Examples of isolated semen may include but are not limited to straws of semen, frozen straws of semen, and semen suitable for use in IVF procedures.

As used herein the terms “polled” preferably refers to the phenotype of an animal which does not possess horns due to it's genotype, when evaluated in a species which may contain horns. Animals that are genetically predisposed to having horns but have been treated to remove or prevent growth of horns are not considered polled, even though they do not possess horns.

As used herein, the term “genetic profile” (GP) refers to a plurality of allelic states of genetic markers characteristic of at least one phenotypic trait for a given animal. Preferably, a genetic profile refers to the allelic state of at least five genetic markers. Various genetic markers, desirable alleles, and genetic profiles are specified below in combination with the Tables and Sequence listing.

As used herein, the term “preferred allele” refers to an allele which is associated with desirable characteristics. A list of specific preferred alleles which are relevant to various embodiments of this invention can be found in Tables 1 and 2.

As used herein, the term “genetic marker” preferably refers to any stable and inherited variation in DNA that can be measured or detected by a suitable method. Genetic markers can be used to detect the presence of a specific genotype or phenotype other than itself, which is otherwise not measurable or very difficult to detect. Examples of genetic markers include, but are not limited to, Single Nucleotide Polymorphism (SNP), Restriction Fragment Length Polymorphism (RFLP), Amplified Fragment Length Polymorphism (AFLP), Copy Number Variation (CNV), Simple Sequence Repeat (SSR, also called microsatellite) and insertions/deletions.

As used herein the terms “animal” or “animals” preferably refer to dairy or beef cattle.

As used herein “fitness” preferably refers to traits that include, but are not limited to: pregnancy rate (PR), daughter pregnancy rate (DPR), productive life (PL), somatic cell count (SCC) and somatic cell score (SCS).

As used herein, PR and DPR refer to the percentage of non-pregnant animals that become pregnant during each 21-day period.

As used herein, PL is analyzed as months in each lactation, summed across all lactations until removal of the cow from the herd (by culling or death).

As used herein, somatic cell score can be calculated using the following relationship: SCS=log₂(SCC/100.000)+3, where SCC is somatic cells per milliliter of milk.

As used herein the term “growth” refers to the measurement of various parameters associated with an increase in an animal's size and/or weight.

As used herein the term “linkage disequilibrium” preferably means allelic association wherein A₁ and B₁ (as used in the above definition of allelic association) are present on the same chromosome.

As used herein the term “marker-assisted selection (MAS) preferably refers to the selection of animals on the basis of marker information in possible combination with pedigree and phenotypic data.

As used herein the term “natural breeding” preferably refers to mating animals without human intervention in the fertilization process. That is, without the use of mechanical or technical methods such as artificial insemination or embryo transfer. The term does not refer to selection of the parent animals.

As used herein the term “net merit” preferably refers to a composite index that includes several commonly measured traits weighted according to relative economic value in a typical production setting and expressed as lifetime economic worth per cow relative to an industry base. Examples of a net merit indexes include, but are not limited to, $NM or TN in the USA, LPI in Canada, etc (formulae for calculating these indices are well known in the art (e.g. $NM can be found on the USDA/AIPL website: www.aipl.arsusda.gov/reference.htm)

As used herein, the term “milk production” preferably refers to phenotypic traits related to the productivity of a dairy animal including milk fluid volume, fat percent, protein percent, fat yield, and protein yield.

As used herein the term “predicted value” preferably refers to an estimate of an animal's breeding value or transmitting ability based on its genotype and pedigree.

As used herein “productivity” and “production” preferably refers to yield traits that include, but are not limited to: total milk yield, milk fat percentage, milk fat yield, milk protein percentage, milk protein yield, total lifetime production, milking speed and lactation persistency.

As used herein the term “quantitative trait” is used to denote a trait that is controlled by multiple (two or more, and often many) genes each of which contributes small to moderate effect on the trait. The observations on quantitative traits often follow a normal distribution.

As used herein the term “quantitative trait locus (QTL)” is used to describe a locus that contains polymorphism that has an effect on a quantitative trait.

As used herein the term “reproductive material” includes, but is not limited to semen, spermatozoa, ova, embryos, and zygote(s).

As used herein the term “single nucleotide polymorphism” or “SNP” refer to a location in an animal's genome that is polymorphic within the population. That is, within the population some individual animals have one type of base at that position, while others have a different base. For example, a SNP might refer to a location in the genome where some animals have a “G” in their DNA sequence, while others have a “T”.

As used herein the term “whole-genome analysis” preferably refers to the process of QTL mapping of the entire genome at high marker density (i.e. at least about one marker per centimorgan) and detection of markers that are in population-wide linkage disequilibrium with QTL.

As used herein the term “whole-genome selection (WGS)” preferably refers to the process of marker-assisted selection (MAS) on a genome-wide basis in which markers spanning the entire genome at moderate to high density (e.g. at least about one marker per 1-5 centimorgans), or at moderate to high density in QTL regions, or directly neighboring or flanking QTL that explain a significant portion of the genetic variation controlling one or more traits.

### ILLUSTRATIVE EMBODIMENTS OF THE INVENTION

Various embodiments of the present invention provide methods for evaluating the genetic profile of a dairy animal or bovine product. In preferred embodiments of the invention, the animal's genotype is evaluated at 12 or more positions (i.e. with respect to 12 or more genetic markers). Aspects of these embodiments of the invention provide methods that comprise determining the animal's genomic sequence at 10 or more locations (loci) that contain single nucleotide polymorphisms (SNPs). Specifically, the invention provides methods for evaluating an animal's genotype by determining which of two or more alleles for the SNP are present for each of 12 or more SNPs selected from the group consisting of the SNPs described in Tables 1 and 2 and the Sequence Listing.

Various embodiments of the invention provide methods for allocating a bovine animal for use according to the animal's genetic profile, the method comprising: a) determining the animal's genotype at 12 or more loci, wherein each locus contains a single nucleotide polymorphism (SNP) having at least two allelic variants; and wherein at least 12 SNPs are selected from the SNPs described in Table 2 and the Sequence Listing; b) analyzing the determined genotype of the at least one evaluated animal; and c) allocating the animal or use based on it's determined genetic profile; wherein the animal is homozygous for the preferred allele for at least 12 SNPs selected from the SNPs described in Table 2 and the Sequence Listing.

Alternative embodiments of this invention include methods wherein part “a)” further comprises determining the animal's genotype at one or more additional loci with each of these additional loci containing at least one additional SNP that has at least two allelic variants; where the additional SNP(s) is/are associated with the polled trait and is/are selected from the SNPs described in Table 1 and the sequence listing; and where the animal is heterozygous for one or more of these additional SNPs.

Alternative aspects of these embodiments of the invention include methods wherein part “a)” further comprises determining the animal's genotype at one or more additional loci with each additional locus containing at least one additional SNP having at least two allelic variants; where the additional SNP(s) is/are associated with the polled trait and is/are selected from the SNPs described in Table 1 and the sequence listing; and where the animal is homozygous for one or more of these additional SNPs.

Yet other alternative aspects of these embodiments of the invention include methods wherein the animal is homozygous for the preferred allele at each of at least about 13, 14, 15, 16, 17, 18, 19, 20, 21, 22, 23, 24, and/or 25 SNPs selected from the SNPs described in Table 2 and the sequence listing. Preferred embodiments of this invention also include methods wherein the animal is polled.

Various embodiments of the invention provide methods for allocating a potential parent bovine animal for use according to the animal's genetic profile. Various aspects of these embodiments comprise: a) determining the animal's genotype at 12 or more loci, wherein each locus contains a single nucleotide polymorphism (SNP) having at least two allelic variants; and wherein at least 12 SNPs are selected from the SNPs described in Table 2 and the Sequence Listing; b) analyzing the determined genotype of the at least one evaluated animal; and c) allocating at least one animal for breeding use based on it's genotype; wherein the animal is homozygous for the preferred allele for at least 12 SNPs selected from the SNPs described in Table 2 and the sequence listing.

Alternative aspects of these embodiments of the invention include methods wherein part “a)” further comprises determining the animal's genotype at one or more additional loci with each additional locus containing at least one additional SNP having at least two allelic variants; where the additional SNP(s) is/are associated with the polled trait and is/are selected from the SNPs described in Table 1 and the sequence listing; and where the animal is heterozygous for one or more of these additional SNPs.

Other aspects of these embodiments of this invention include methods wherein part “a)” further comprises determining the animal's genotype at one or more additional loci with each additional locus containing at least one additional SNP having at least two allelic variants where the additional SNP(s) is/are associated with the polled trait and is/are selected from the SNPs described in Table 1 and the sequence listing; and where the animal is homozygous for the one or more of these additional SNPs.

Still other aspects of these embodiments of the invention include methods wherein the animal is homozygous for the preferred allele at each of at least 13, 14, 15, 16, 17, 18, 19, 20, 21, 22, 23, 24, and/or 25 SNPs selected from the SNPs described in Table 2 and the sequence listing. Preferred embodiments of this invention also include methods wherein the animal is polled.

Other embodiments of the invention provide methods for producing progeny from bovine animals, the methods comprising: a) identifying at least one potential parent animal that has been allocated for breeding in accordance with any of the methods described herein; b) producing progeny from the allocated animal through a process selected from the group consisting of: (i) natural breeding; (ii) artificial insemination; (iii) in vitro fertilization; and c) collecting semen/spermatozoa or at least one ovum from the animal and contacting it, respectively, with ovum/ova or semen/spermatozoa from a second animal to produce a conceptus by any means.

Alternative aspects of these embodiments of the invention include methods comprising producing progeny through natural breeding.

Other aspects of these embodiments of the invention include methods which include producing offspring through artificial insemination, embryo transfer, and/or in vitro fertilization.

Other embodiments of the invention provide for bovine products having genetic profiles wherein the genetic profile comprises single nucleotide polymorphisms (SNPs); wherein the product comprises at least 12 SNPs selected from the SNPs described in Table 2 and the Sequence Listing; and wherein the product is homozygous for the preferred allele of at least 12 of the SNPs described in Table 2.

Certain aspects of these embodiments of the invention include a bovine product that is heterozygous for at least one allele associated with the polled trait for at least one SNP selected from the SNPs described in Table 1.

Other aspects of these embodiments of the invention include a bovine product that is homozygous for at least one allele associated with the polled trait for at least one SNP selected from the SNPs described in Table 1.

Still other aspects of these embodiments of the invention include methods wherein the bovine product is homozygous for the preferred allele at each of at least about 13, 14, 15, 16, 17, 18, 19, 20, 21, 22, 23. 24. and/or 25 SNPs selected from the SNPs described in Table 2 and the sequence listing.

Preferred aspects of these embodiments of the invention include bovine products wherein the bovine product is isolated semen.

Other embodiments of the invention provide bovine animal(s) having a genetic profile wherein the genetic profile comprises single nucleotide polymorphisms (SNPs); where the animal comprises at least 12 SNPs selected from the SNPs described in Table 2 and the Sequence Listing; and where the animal is homozygous for the preferred allele of at least 12 of the SNP described in Table 2.

Alternative aspects of these embodiments of the invention provide for a bovine animal that is heterozygous for at least one allele associated with the polled trait for at least one SNP selected from the SNPs described in Table 1.

Other aspects of these embodiments of the invention provide a bovine animal that is homozygous for at least one allele associated with the polled trait for at least one SNP selected from the SNPs described in Table 1.

Still other aspects of these embodiments of the invention provide a bovine animal wherein the bovine animal is homozygous for the preferred allele at each of at least 13, 14, 15, 16, 17, 18, 19, 20, 21, 22, 23, 24, and/or 25 SNPs selected from the SNPs described in Table 2 and the sequence listing.

Particularly preferred aspects of these embodiments of the invention provide for polled bovine animals.

Various Embodiments of the invention provide methods for determining a genetic profile of a bovine product, the methods comprising: a) collecting a sample of biological material containing DNA; b) determining the genotype of the biological material at 12 or more loci, wherein each locus contains a single nucleotide polymorphism (SNP) having at least two allelic variants: where at least 12 SNPs are selected from the SNPs described in Table 2 and the Sequence Listing; and c) analyzing the determined genotype; wherein the biological material is homozygous for the preferred allele for at least 12 SNPs selected from the SNPs described in Table 2 and the Sequence Listing.

Alternative aspects of these embodiments of the invention provide methods wherein step “b)” further comprises determining the genotype of the biological material at one or more additional loci, with each additional locus containing at least on additional SNP having at least two allelic variants; where (i) the additional SNP is selected from the SNPs described in Table 1 and the sequence listing; and (ii) the biological material is heterozygous for at least one allele associated with the polled trait as described in Table 1.

Other aspects of these embodiments of the invention provide methods wherein step “b)” further comprises determining the genotype of the biological material at one or more additional loci with each additional locus containing at least one additional SNP having at least two allelic variants; where (i) the SNP is selected from the SNPs described in Table 1 and the sequence listing; and (ii) the biological material is homozygous for at least one allele associated with the polled trait as described in Table 1.

In preferred embodiments of the invention the animal's genotype is evaluated to determine which allele is present for SNPs selected from the group of SNPs described in Table 1 and/or Table 2 and the Sequence Listing.

In any embodiments of the invention, the animal's genotype may be analyzed with respect to SNPs that have been shown to be associated with one or more traits (see Table 1) and are used to calculate a genetic profile. For example, embodiments of the invention provides a method for genotyping 10 or more, 25 or more, 50 or more, 100 or more, 200 or more, or 500 or more, or 1000 or more SNPs that have been determined to be significantly associated with one or more of these traits. At least two of these SNPs are preferably selected from the group consisting of the SNPs described in Table 1 and the Sequence Listing

Various embodiments of the present invention also provide for both whole-genome analysis and whole genome-selection (WGS) (i.e. marker-assisted selection (MAS) on a genome-wide basis). Moreover, in any aspect of these embodiments of the invention the markers used to carry out the whole-genome analysis may include one or more markers that are selected from the group consisting of the markers described in Table 1 and the Sequence Listing.

In any embodiment of the invention the genomic sequence at the SNP locus may be determined by any means compatible with the present invention. Suitable means are well known to those skilled in the art and include, but are not limited to direct sequencing, sequencing by synthesis, primer extension, Matrix Assisted Laser Desorption/Ionization-Time Of Flight (MALDI-TOF) mass spectrometry, polymerase chain reaction-restriction fragment length polymorphism, microarray/multiplex array systems (e.g. those available from Illumina Inc., San Diego, Calif. or Affymetrix, Santa Clara, Calif.), and allele-specific hybridization.

Other embodiments of the invention provide methods for allocating animals for subsequent use (e.g. to be used as sires or dams or to be sold for meat or dairy purposes) according to their predicted value for horned/polled, productivity, or fitness. Various aspects of this embodiment of the invention comprise determining at least one animal's genotype for at least one SNP selected from the group of SNPs consisting of the SNPs described in Table 1 and the sequence listing, (methods for determining animals' genotypes for one or more SNPs are described supra). Thus, the animal's allocation for use may be determined based on its genotype and resulting genetic profile.

The instant invention also provides embodiments where analysis of the genotypes of the SNPs described in Table 1 and the Sequence Listing is the only analysis done. Other embodiments provide methods where analysis of the SNPs disclosed herein is combined with any other desired type of genomic or phenotypic analysis (e.g. analysis of any genetic markers beyond those disclosed in the instant invention).

According to various aspects of these embodiments of the invention, once the animal's genetic sequence for the selected SNP(s) have been determined, this information is evaluated to determine which allele of the SNP is present for selected SNPs. Preferably, the animal's allelic complement for all of the determined SNPs is evaluated. Next, a genetic profile is analyzed based on specific methods described below. Finally, the animal is allocated for use based on its genotype for one or more of the SNP positions evaluated. Preferably, the allocation is made taking into account the animal's genetic profile.

The allocation may be made based on any suitable criteria. For any genetic profile, a determination may be made as to whether an animal's genetic profile exceeds target values. This determination will often depend on breeding or herd management goals. Additionally, other embodiments of the invention provide methods where combinations of two or more criteria are used. Such combinations of criteria include but are not limited to, two or more criterion selected from the group consisting of: phenotypic data, pedigree information, breed information, the animal's genetic profile, and genetic profile information from siblings, progeny, and/or parents.

Determination of which alleles are associated with desirable phenotypic characteristics can be made by any suitable means. Methods for determining these associations are well known in the art; moreover, aspects of the use of these methods are generally described in the EXAMPLES, below.

According to various aspects of this embodiment of the invention allocation for use of the animal may entail either positive selection for the animals having the desired genetic profile (e.g. the animals with the desired genotypes are selected), negative selection of animals having an undesirable genetic profile, or any combination of these methods.

According to preferred aspects of this embodiment of the invention, animals or bovine products identified as having a genetic profile above a minimum threshold are allocated to a use consistent with animals having higher economic value. Alternatively, animals or bovine products that have a genetic profile lower than the minimum threshold are not allocated for the same use as those with a higher genetic profile.

Other embodiments of the invention provide methods for selecting potential parent animals (i.e., allocation for breeding) to improve fitness and/or productivity in potential offspring. Various aspects of this embodiment of the invention comprise determining at least one animal's genetic profile using SNPs selected from the group of SNPs consisting of the SNPs described in Table 1 and Table 2 and the Sequence Listing. Furthermore, determination of whether and how an animal will be used as a potential parent animal may be based on its genetic profile, pedigree information, breed information, phenotypic information, progeny information, or any combinations thereof.

Moreover, as with other types of allocation for use, various aspects of these embodiments of the invention provide methods where the only analysis done is to analyze the genetic profile. Other aspects of these embodiments provide methods where analysis of the genetic profile disclosed herein is combined with any other desired genomic or phenotypic analysis (e.g. analysis of any genetic markers beyond those disclosed in the instant invention).

According to various aspects of these embodiments of the invention, once the animal's genetic sequence at the site of the selected SNP(s) have been determined, this information is evaluated to determine which allele of the SNP is present for at least one of the selected SNPs. Preferably the animal's allelic complement for all of the sequenced SNPs is evaluated. Additionally, the animal's allelic complement is analyzed and evaluated to analyze the genetic profile and thereby predict the animal's progeny's genetic merit or phenotypic value. Finally, the animal is allocated for use based on its genetic profile, either alone or in combination with one or more additional criterion/criteria.

Other embodiments of the instant invention provide methods for producing progeny animals. According to various aspects of this embodiment of the invention, the animals used to produce the progeny are those that have been allocated for breeding according to any of the embodiments of the current invention. Those using the animals to produce progeny may perform the necessary analysis or, alternatively, those producing the progeny may obtain animals that have been analyzed by another. The progeny may be produced by any appropriate means, including, but not limited to using: (i) natural breeding, (ii) artificial insemination, (iii) in vitro fertilization (IVF) or (iv) collecting semen/spermatozoa and/or at least one ovum from the animal and contacting it, respectively with ova/ovum or semen/spermatozoa from a second animal to produce a conceptus by any means.

According to other aspects of the invention, the progeny are produced through a process comprising the use of standard artificial insemination (AI), in vitro fertilization, multiple ovulation embryo transfer (MOET), or any combination thereof.

Other embodiments of the invention provide for methods that comprise allocating an animal for breeding purposes and collecting/isolating genetic material from that animal: wherein genetic material includes but is not limited to: semen, spermatozoa, ovum, zygotes, blood, tissue, serum, DNA, and RNA,

It is understood that most efficient and effective use of the methods and information provided by the instant invention employ computer programs and/or electronically accessible databases that comprise or a portion of the sequences disclosed in the instant application. Accordingly, the various embodiments of the instant invention provide for databases comprising all or a portion of the sequences corresponding to at least 12 SNPs described in Table 1 and Table 2 and the Sequence Listing. In preferred aspect of these embodiments the databases comprise sequences for 25 or more, 50 or more, 100 or more SNPs, at least one of which are SNPs described in Table 1 and the Sequence Listing.

It is further understood that efficient analysis and use of the methods and information provided by the instant invention will employ the use of automated genotyping. Any suitable method known in the art may be used to perform such genotyping, including, but not limited to the use of micro-arrays.

Other embodiments of the invention provide methods wherein one or more of the SNP sequence databases described herein are accessed by one or more computer-executable programs. Such methods include, but are not limited to, use of the databases by programs to analyze for an association between the SNP and a phenotypic trait, or other user-defined trait (e.g. traits measured using one or more metrics such as gene expression levels, protein expression levels, or chemical profiles), calculation of a genetic profile, and programs used to allocate animals for breeding or market.

Other embodiments of the invention provide methods comprising collecting genetic material and calculating a genetic profile from an animal that has been allocated for breeding. Wherein the animal has been allocated for breeding by any of the methods disclosed as part of the instant invention.

Other embodiments of the invention provide for diagnostic kits or other diagnostic devices for determining which allele of one or more SNP(s) is/are present in a sample: wherein the SNP(s) are selected from the group of SNPs consisting of the SNPs described in Table 1 and the sequence listing. In various aspects of this embodiment of the invention, the kit or device provides reagents/instruments to facilitate a determination as to whether nucleic acid corresponding to the SNP is present. Such kit/or device may further facilitate a determination as to which allele of the SNP is present. In certain aspects of this embodiment of the invention the kit or device comprises at least one nucleic acid oligonucleotide suitable for DNA amplification (e.g. through polymerase chain reaction). In other aspects of the invention the kit or device comprises a purified nucleic acid fragment capable of specifically hybridizing, under stringent conditions, with at least one allele of at least ten of the SNPs described in Table 1 and the Sequence listing.

In particularly preferred aspects of this embodiment of the invention the kit or device comprises at least one nucleic acid array (e.g. DNA micro-arrays) capable of determining which allele of one or more of the SNPs are present in a sample; where the SNPs are selected from the group of SNPs consisting of the SNPs described in Table 1 and the Sequence Listing. Preferred aspects of this embodiment of the invention provide DNA micro-arrays capable of simultaneously determining which allele is present in a sample for 10 or more SNPs. Preferably, the DNA micro-array is capable of determining which SNP allele is present in a sample for 25 or more, 50 or more, 100 or more SNPs. Methods for making such arrays are known to those skilled in the art and such arrays are commercially available (e.g. from Affymetrix, Santa Clara, Calif.).

Genetic markers that are in allelic association with any of the SNPs described in the Tables may be identified by any suitable means known to those skilled in the art. For example, a genomic library may be screened using a probe specific for any of the sequences of the SNPs described in the Tables. In this way clones comprising at least a portion of that sequence can be identified and then up to 300 kilobases of 3′ and/or 5′ flanking chromosomal sequence can be determined. Preferably up to about 70 kilobases of 3′ and/or 5′ flanking chromosomal sequences are evaluated. By this means, genetic markers in allelic association with the SNPs described in the Tables will be identified. These alternative markers in allelic association may be used to select animals in place of the markers described in Table 1 and the sequence listing.

In preferred embodiments of the invention, a genetic profile is analyzed based on genotypic information acquired from a dairy animal or bovine product. The genetic profile has been created using information from the whole genome genetic analysis described above. SNP discovery techniques, and candidate gene analysis. The profile was created using the trait association, effect estimates, and expected values of the underlying markers.

Other embodiments of the invention provide isolated semen comprising improved genetic content. Preferably, the isolated semen comprising improved genetic content further comprise improved genetic profiles as described herein. Various embodiments of the invention also comprise frozen isolated semen, and isolated semen with disproportionate sex determining characteristics, such as for example, greater than naturally occurring frequencies of X chromosomes.

When determining the genetic profile of sperm or semen, the genetic profile is determined based on all alleles present in the source animal for each SNP, including those homozygous for each allele and heterozygous for combinations of alleles. Because each individual sperm and unfertilized egg contains only a haploid genome (as opposed to a genome), the genetic profile calculations provided herein are only applicable in those instances where a sufficient number of haploid cells are present to determine the diploid genotype of the animal from which the cells were derived (i.e. greater than about 50 individual cells).

When determining the genetic profile of other bovine products, at least one DNA sample must be retrieved from the product. For example, when testing milk, DNA may be retrieved from the leucocytes cells contained therein. When testing bovine meat products. DNA can be extracted from the muscle fibers. Preferably when evaluating the genetic profile of bovine products. DNA from at least about 50 individual cells are used to determine the genetic profile. However, recent advances in the field of DNA extraction and replication allow for determining genetic content from a sample as small as one cell (Zhang, 2006).

Methods of collecting, storing, freezing, and using isolated semen are well known in the art. Any suitable techniques can be utilized in conjunction with the genetic profiles described herein. Furthermore, techniques for altering sex determining characteristics such as the frequency of X chromosomes in the sperm suspension are also known. A variety of methods for altering sex determining characteristics are known in the art, including for example, cell cytometry, photodamage, and microfluidics. The following references related to methods of collecting, storing, freezing, and altering sex-determining characteristics of sperm suspensions are herby incorporated by reference: U.S. Pat. No. 5,135,759, U.S. Pat. No. 5,985,216, U.S. Pat. No. 6,071,689, U.S. Pat. No. 3,614,9867, U.S. Pat. No. 6,263,745, U.S. Pat. No. 6,357,307, U.S. Pat. No. 6,372,422, U.S. Pat. No. 6,524,860, U.S. Pat. No. 3,660,4435, U.S. Pat. No. 6,617,107, U.S. Pat. No. 6,746,873, U.S. Pat. No. 6,782,768, U.S. Pat. No. 6,819,411, U.S. Pat. No. 7,094,527, U.S. Pat. No. 3,7169,548, US2002005076A1, US2002096123A1, US2002119558A1, US2002129669A1, US2003157475A1, US2004031071A1, US2004049801A1, US2004050186A1, US2004053243A1, US2004055030A1, US2005003472A1, US2005112541A1, US2005130115A1, US2005214733A1, US2005244805A1, US2005282245A1, US2006067916A1, US2006118167A1, US2006121440A1, US2006141628A1, US2006170912A1, US2006172315A1, US2006229367A1, US2006263829A1, US2006281176A1, US2007026378A1. US2007026379A1, US2007042342A1.

### Examples

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

### Example 1

### Determining Associations Between Genetic Markers and Phenotypic Traits or Profiles

Simultaneous discovery and fine-mapping on a genome-wide basis of genes underlying quantitative traits (Quantitative Trait Loci: QTL) requires genetic markers densely covering the entire genome. As described in this example, a whole-genome, dense-coverage marker map was constructed from microsatellite and single nucleotide polymorphism (SNP) markers with previous estimates of location in the bovine genome, and from SNP markers with putative locations in the bovine genome based on homology with human sequence and the human/cow comparative map. A new linkage-mapping software package was developed, as an extension of the CRIMAP software (Green et al., Washington University School of Medicine, St. Louis, 1990), to allow more efficient mapping of densely-spaced markers genome-wide in a pedigreed livestock population (Liu and Grosz Abstract C014; Grapes et al. Abstract W244; 2006 Proceedings of the XIV Plant and Animal Genome Conference, www.intl-pag.org). The new linkage mapping tools build on the basic mapping principles programmed in CRIMAP to improve efficiency through partitioning of large pedigrees, automation of chromosomal assignment and two-point linkage analysis, and merging of sub-maps into complete chromosomes. The resulting whole-genome discovery map (WGDM) included 6,966 markers and a map length of 3,290 centimorgans (cM) for an average map density of 2.18 markers/cM. The average gap between markers was 0.47 cM and the largest gap was 7.8 cM. This map provided the basis for whole-genome analysis and fine-mapping of QTL contributing to variation in productivity and fitness in dairy cattle.

### Discovery and Mapping Populations

Systems for discovery and mapping populations can take many forms. The most effective strategies for determining population-wide marker/QTL associations include a large and genetically diverse sample of individuals with phenotypic measurements of interest collected in a design that allows accounting for non-genetic effects and includes information regarding the pedigree of the individuals measured. In the present example, an outbred population following the grand-daughter design (Weller et al., 1990) was used to discover and map QTL: the population, from the Holstein breed, had 529 sires each with an average of 6.1 genotyped sons, and each son has an average of 4216 daughters with milk data. DNA samples were collected from approximately 3,200 Holstein bulls and about 350 bulls from other dairy breeds; representing multiple sire and grandsire families.

### Phenotypic Analyses

Dairy traits under evaluation include traditional traits such as milk yield (“MILK”) (pounds), fat yield (“FAT”) (pounds), fat percentage (“FATPCT”) (percent), productive life (“PL”) (months), somatic cell score (“SCS”) (Log), daughter pregnancy rate (“DPR”) (percent), protein yield (“PROT”) (pounds), protein percentage (“PROTPCT”) (percent), and net merit (“NM”) (dollar), and combinations of multiple traits, such as for example in a genetic profile. These traits are sex-limited, as no individual phenotypes can be measured on male animals. Instead, genetic merits of these traits defined as PTA (predicted transmitting ability) were estimated using phenotypes of all relatives. Most dairy bulls were progeny tested with a reasonably larger number of daughters (e.g., >50), and their PTA estimation is generally more or considerably more accurate than individual cow phenotype data. The genetic evaluation for traditional dairy traits of the US Holstein population is performed quarterly by USDA. Detailed descriptions of traits, genetic evaluation procedures, and genetic parameters used in the evaluation can be found at the USDA AIPL web site (www.aipl.arsusda.gov). It is meaningful to note that the dairy traits evaluated in this example are not independent: FAT and PROT are composite traits of MILK and FATPCT, and MILK and PROTPCT, respectively. NM is an index trait calculated based on protein yield, fat yield, production life, somatic cell score, daughter pregnancy, calving difficulty, and several type traits. Protein yield and fat yield together account for >50% of NM, and the value of milk yield, fat content, and protein content is accounted for via protein yield and fat yield.

PTA data of all bulls with progeny testing data were downloaded from the USDA evaluation published at the AIPL site in February 2007. The PTA data were analyzed using the following two models:

yᵢⱼ=sᵢ+PTAdᵢⱼ [Equation 4]

yᵢ=β+β₁(SPTA)ᵢ+PTAdᵢ [Equation 5]

where yᵢ (yᵢⱼ) is the PTA of the i^{th} bull (PTA of the j^{th} son of the i^{th} sin); sᵢ is the effect of the i^{th} sire; (SPTA)ᵢ is the sire's PTA of the i^{th} bull of the whole sample; μ is the population mean; PTAdᵢ (PTAdᵢⱼ) is the residual bull PTA.

Equation 4 is referred to as the sire model, in which sires were fitted as fixed factors. Among all USA Holstein progeny tested bulls, a considerably large number of sires only have a very small number of progeny tested sons (e.g., some have one son), and it is clearly undesirable to fit sires as fixed factors in these cases. It is well known the USA Holstein herds have been making steady and rapid genetic progress in traditional dairy traits in the last several decades, implying that the sire's effect can be partially accounted for by fitting the birth year of a bull. For sires with <10 progeny tested sons, sires were replaced with son's birth year in Equation 4. Equation 5 is referred to as the SPTA model, in which sire's PTA are fitted as a covariate. Residual PTA (PTAdᵢ or PTAdᵢⱼ) were estimated using linear regression.

### Example 2

### Use of Single Nucleotide Polymorphisms and Genetic Profiles to Improve Offspring Traits

To improve the average genetic merit of a population for a chosen trait, one or more of the markers with significant association to that trait can be used in selection of breeding animals. In the case of each discovered locus, use of animals possessing a marker allele (or a haplotype of multiple marker alleles) in population-wide Linkage Disequilibrium (LD) with a favorable QTL allele will increase the breeding value of animals used in breeding, increase the frequency of that QTL allele in the population over time and thereby increase the average genetic merit of the population for that trait. This increased genetic merit can be disseminated to commercial populations for full realization of value.

Furthermore, multiple markers can be used simultaneously, such as for example, when improving offspring traits using a genetic profile. In this case, a plurality of markers are measured and weighted according to the value of the associated traits and the estimated effect of the marker on the trait. The development of a preferred genetic profile allows inclusion of multiple traits and markers simultaneously, thereby optimizing multiple parameters of the selection process.

For example, a DNA-testing program scheme could greatly change the frequency of the polled allele in a given population or semen product via the use of DNA markers for screening bulls as described herein. Testing semen from bulls within a progeny testing program would identify the genotype of the bull at the horned/polled locus. This information creates value because this knowledge influences market desirability of the semen product. Typically, a progeny testing program uses pedigree information and performance of relatives to select juvenile bulls as candidates for entry into the program. However, by adding horned/polled marker information, young bulls could be screened to identify those animals carrying (or homozygous for) the polled marker/allele. The use of these animals to create the next generation of animals would not only create more naturally polled animals (since polled is dominant), but would also increase the frequency of the polled allele in the population from which the next generation of parents will ultimately be selected.

Additionally. DNA samples from potential bull mothers and their male offspring could be screened with markers from Table 1, and bull-mother candidates with preferable genotypes can be contracted for matings to tested bulls. If superovulation and embryo transfer (ET) is employed, a set of 5-10 offspring could be produced per bull mother per flush procedure. Then the markers could again be used to select a polled male offspring as a candidate for the progeny test program, or a female offspring as a future bull mother.

The first step in using a SNP for estimation of breeding value and selection in the genetic nucleus (GN) is collection of DNA from all offspring that will be candidates for selection as breeders in the GN or as breeders in other commercial populations. One method is to capture shortly after birth a small bit of ear tissue, hair sample, or blood from each calf into a labeled (bar-coded) tube. Another method is to directly test semen from bulls available for breeding. The DNA extracted from this tissue can be used to assay an essentially unlimited number of SNP markers, including the horned/polled markers described in Table 1, and the results can be included in selection decisions before the animal reaches breeding age.

The markers described herein can be used in breeding schemes in combination with markers that are associated with phenotypic traits of economic relevance. One method for incorporating into selection decisions the markers (or marker haplotypes) determined to be in population-wide LD with valuable QTL alleles is based on classical quantitative genetics and selection index theory (Falconer and Mackay, 1996; Dekkers and Chakrabony, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker.

Alternatively, a set of markers associated with phenotypic traits could be used to create a genetic profile, and the bull-mother candidates with genetic profiles above pre-determined thresholds could be contracted for matings to specific bulls. Furthermore, combinations of genetic profiles, associated markers, phenotypic data, pedigree information, and other historical performance parameters can be used simultaneously.

If superovulation and embryo transfer (ET) is employed, a set of 5-10 offspring could be produced per bull mother per flush procedure. Then the marker set could again be used to select the best male offspring as a candidate for the progeny test program. If genome-wide markers are used, it was estimated that accuracies of marker selection could reach as high as 0.85 (Meuwissen et al., 2001). This additional accuracy could be used to greatly improve the genetic merit of candidates entering the progeny test program and thereby increasing the probability of successfully graduating a marketable progeny-tested bulls. This information could also be used to reduce program costs by decreasing the number of juvenile bull candidates tested while maintaining the same number of successful graduates. In the extreme, very accurate Genetic profiles (genetic profiles) could be used to directly market semen from juvenile sires without the need of progeny-testing at all. Due to the fact that juveniles could now be marketed starting at puberty instead of 4.5 to 5 years, generation interval could be reduced by more than half and rates of gain could increase as much as 68.3% (Schrooten et al., 2004). With the elimination of the need for progeny testing, the cost of genetic improvement for the artificial insemination industry would be vastly improved (Schaeffer, 2006).

In an alternate example, a centralized or dispersed genetic nucleus (GN) population of cattle could be maintained to produce juvenile bulls for use in progeny testing or direct sale on the basis of genetic profiles. A GN herd of 1000 cows could be expected to produce roughly 3000 offspring per year, assuming the top 10-15% of females were used as ET donors in a multiple-ovulation and embryo-transfer (MOET) scheme. However, markers could change the effectiveness of MOET schemes and in vitro embryo production. Previously, MOET nucleus schemes have proven to be promising from the standpoint of extra genetic gain, but the costs of operating a nucleus herd together with the limited information on juvenile animals has limited widespread adoption. However, with marker information and/or genetic profiles, juveniles can be selected much more accurately than before resulting in greatly reduced generation intervals and boosted rates of genetic response. This is especially true in MOET nucleus herd schemes because, previously, breeding values of full-sibs would be identical, but with marker information the best full-sib can be identified early in life. The marker information and/or genetic profile would also help limit inbreeding because less selection pressure would be placed on pedigree information and more on individual marker information. An early study (Meuwissen and van Arendonk, 1992) found advantages of up to 26% additional genetic gain when markers were employed in nucleus herd scenarios: whereas, the benefit in regular progeny testing was much less.

Together with MAS, female selection could also become an important source of genetic improvement particularly if markers explain substantial amounts of genetic variation. Further efficiencies could be gained by marker testing of embryos prior to implantation (Bredbacka, 2001). This would allow considerable selection to occur on embryos such that embryos with inferior marker profiles could be discarded prior to implantation and recipient costs. This would again increase the cost effectiveness of nucleus herds because embryo pre-selection would allow equal progress to be made with a smaller nucleus herd. Alternatively, this presents further opportunities for pre-selection prior to bulls entering progeny test and rates of genetic response predicted to be up to 31% faster than conventional progeny testing (Schrooten et al., 2004).

The first step in using a genetic profile for estimation of breeding value and selection in the GN is collection of DNA from all offspring that will be candidates for selection as breeders in the GN or as breeders in other commercial populations (in the present example, the 3,000 offspring produced in the GN each year). One method is to capture shortly after birth a small bit of ear tissue, hair sample, or blood from each calf into a labeled (bar-coded) tube. The DNA extracted from this tissue can be used to assay a large number of SNP markers. Then the animal's genetic profile can be calculated and the results used in selection decisions before the animal reaches breeding age.

One method for incorporating into selection decisions the markers (or marker haplotypes) determined to be in population-wide LD with valuable QTL alleles (see Example 1) is based on classical quantitative genetics and selection profile theory (Falconer and Mackay, 1996; Dekkers and Chakraborty, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker:

α₁=q[a+d(q−p)] [Equation 6]

α₂=−p[a+d(q−p)] [Equation 7]

α=a+d(q−p) [Equation 8]

g_{A1A1}=2(α₁) [Equation 9]

g_{A1A2}=(α₁)+(α₂) [Equation 10]

g_{A2A2}=2(α₂) [Equation 11]

where α₁ and α₂ are the average effects of alleles 1 and 2, respectively: a is the average effect of allele substitution; p and q are the frequencies in the population of alleles 1 and 2, respectively; a and d are additive and dominance effects, respectively; g_{A1A1}, g_{A1A2} and g_{A2A2} are the (marker) breeding values for animals with marker genotypes A1A1, A1A2 and A2A2, respectively. The total trait breeding value for an animal is the sum of breeding values for each marker (or haplotype) considered and the residual polygenic breeding value:

EBVᵢⱼ=Σ ĝⱼ+Ûᵢ [Equation 12]

where EBVᵢⱼ is the Estimated Trait Breeding Value for the i^{th} animal, Σ ĝⱼ is the marker breeding value summed from j=1 to n where n is the total number of markers (haplotypes) under consideration, and Ûᵢ is the polygenic breeding value for the i^{th} animal after fitting the marker genotype(s).

These methods can readily be extended to estimate breeding values for selection candidates for multiple traits including genetic profiles. The breeding value for each trait including information from multiple markers (haplotypes), are all within the context of selection profile theory and specific breeding objectives that set the relative importance of each trait. Other methods also exist for optimizing marker information in estimation of breeding values for multiple traits, including random models that account for recombination between markers and QTL (e.g., Fernando and Grossman, 1989), and the potential inclusion of all discovered marker information in whole-genome selection (Meuwissen et al., Genetics 2001). Through any of these methods, the markers reported herein that have been determined to be in population-wide LD with valuable QTL alleles may be used to provide greater accuracy of selection, greater rate of genetic improvement, and greater value accumulation in the dairy industry.

### Example 3

### Identification of SNPs

A nucleic acid sequence contains a SNP of embodiments of present invention if it comprises at least 20 consecutive nucleotides that include and/or are adjacent to a polymorphism described in Table 1 or 2 and the Sequence Listing. Alternatively, a SNP may be identified by a shorter stretch of consecutive nucleotides which include or are adjacent to a polymorphism which is described in Table 1 or 2 and the Sequence Listing in instances where the shorter sequence of consecutive nucleotides is unique in the bovine genome. A SNP site is usually characterized by the consensus sequence in which the polymorphic site is contained, the position of the polymorphic site, and the various alleles at the polymorphic site. “Consensus sequence” means DNA sequence constructed as the consensus at each nucleotide position of a cluster of aligned sequences.

Such SNP have a nucleic acid sequence having at least 90% sequence identity, more preferably at least 95% or even more preferably for some alleles at least 98% and in many cases at least 99% sequence identity, to the sequence of the same number of nucleotides in either strand of a segment of animal DNA which includes or is adjacent to the polymorphism. The nucleotide sequence of one strand of such a segment of animal DNA may be found in a sequence in the group consisting of SEQ ID NO:1 through SEQ ID NO:46. It is understood by the very nature of polymorphisms that for at least some alleles there will be no identity at the polymorphic site itself. Thus, sequence identity can be determined for sequence that is exclusive of the polymorphism sequence. The polymorphisms in each locus are described in the sequence listing.

Shown below are examples of public bovine SNPs that match each other: SNP ss38333809 was determined to be the same as ss38333810 because 41 bases (with the polymorphic site at the middle) from each sequence match one another perfectly (match length=41, identity=100%).

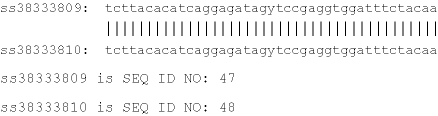

SNP ss38333809 was determined to be the same as ss38334335 because 41 bases (with the polymorphic site at the middle) from each sequence match one another at all bases except for one base (match length=41, identity=97%).

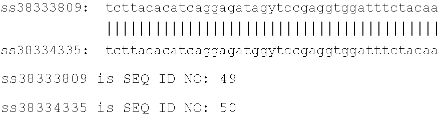

### Example 4

### Quantification of and Genetic Evaluation for Production Traits

Quantifying production traits can be accomplished by measuring milk of a cow and milk composition at each milking, or in certain time intervals only. In the USDA yield evaluation the milk production data are collected by Dairy Herd Improvement Associations (DHIA) using ICAR approved methods. Genetic evaluation includes all cows with the known sire and the first calving in 1960 and later and pedigree from birth year 1950 on. Lactations shorter than 305 days are extended to 305 days. All records are preadjusted for effects of age at calving, month of calving, times milked per day, previous days open, and heterogeneous variance. Genetic evaluation is conducted using the single-trait BLUP repeatability model. The model includes fixed effects of management group (herd×year×season plus register status), parity×age, and inbreeding, and random effects of permanent environment and herd by sire interaction. PTAs are estimated and published four times a year (February, May, August, and November). PTAs are calculated relative to a five year stepwise base i.e., as a difference from the average of all cows born in the current year, minus five (5) years. Bull PTAs are published estimating daughter performance for bulls having at least 10 daughters with valid lactation records.

### Example 5

### Identifying Markers Associated with the Horned/Polled Phenotype in Dairy Cattle

The polled mutation in Bos taurus, which is unknown, was localized to the proximal end of bovine chromosome 1 (BTA01) by Georges et al. (1993) utilizing microsatellite markers. More recent efforts to fine-map the polled locus have included additional microsatellite marker mapping (Schmutz et al. 1995; Brenneman et al. 1996; Harlizius et al. 1997; Drögemüller et al. 2005) and the creation of a BAC-based physical map of the polled region (Wunderlich et al. 200(1). The location of the most proximal gene. ATP5O, and most distal gene. KRTAP8, of the polled region from these cited sources corresponds to approximately 0.6 Mb and 3.9 Mb respectively on the public bovine genome assembly version 3.1 (www.hgsc.bcm.tmc.edu/projects/bovine/).

The objective of this work was to identify single nucleotide polymorphisms (SNPs) associated with the polled trait by sequencing targeted regions of the proximal end of BTA01 on a discovery panel of polled and horned Holsteins.

### Discovery and Mapping Populations

All DNA samples were extracted from spermatozoa using the Qiagen Biosprint (Qiagen Inc., Valencia, Calif.) according to the manufacturer's protocol. Twenty-four Holstein bulls were utilized as a polymorphism discovery panel. Within the set of 24, pairs of animals are directly related as horned sires and polled sons, with the dams expected to be polled based on the reported phenotype of the animal. Semen samples from the 12 polled sons, were obtained from two dairy producers who specifically breed for the polled trait by utilizing polled females. To incorporate the industry elite genetics, top sires are bred to these polled females. Therefore, any polymorphism identified as concordant with the polled/horned trait would be homozygous for one allele in the 12 horned bulls and heterozygous (or infrequently homozygous for the second allele) in the 12 polled bulls. (Assuming that the allele frequency of the polled allele is 0.1, the probability of finding a homozygous polled son in this population can be calculated to be 10%).

### PCR

PCR primers were designed to target gene coding regions and regulatory elements (untranslated regions, putative promoters) including an average of 70 by flanking sequence from target genes within the region. Optimal primer annealing temperatures were obtained by using gradient PCR thermocycling conditions of 15 minutes at 95° C., 35 cycles of 45 seconds at 94° C., 45 seconds of gradient temperatures starting at 55° to 66° across twelve sample wells, 45 seconds at 72° C., and 10 minutes 72° C. Once an optimal annealing temperature was found, each primer set was amplified for sequencing using standard thermocycling conditions of 15 minutes at 95° C. followed by 35 cycles of 45 seconds at 94° C., 45 seconds at optimal annealing temp, and 45 seconds at 72° C., with a final extension step of 10 minutes at 72° C. Concentrations for a 10 microliter PCR volume (gradient and standard) were 5 nanograms per microliter of genomic DNA. 0.5 micromolar of each primer (forward and reverse), 1× SIGMA JumpStart PCR Mix (Sigma-Aldrich Co., St. Louis, Mo.).

### Putative Regulatory Element Prediction

The on-line resource WWW Promoter Scan (www-bimas.cit.nih.gov/molbio/proscan/) was used to scan targeted gene introns and inter-genic sequences for predicted regulatory elements such as promoters and transcription factor binding sites. Identified putative regulatory elements were included with gene coding and regulatory regions (UTRs) for primer design and targeted polymorphism discovery.

### Sequencing

Sequencing was carried out using the method described below and all sequencing was done in both forward and reverse directions. A 10 microliter standard PCR was performed, of which 5 microliter was visualized by agarose gel electrophoresis for confirmation of amplification and the remaining 5 microliter purified using the EXO-SAP-IT PCR Product Clean-up (USB corporation, Cleveland, Ohio) according to the manufacturer's protocol. Direct sequencing of purified PCR products was conducted in 9 microliter reaction volume of 7 microliter of purified PCR product and 2 microliter of 10 micromolar primer, both forward or reverse, and resolved on an ABI 3730x1 Automated Sequencer (Applied Biosystems, Foster City, Calif.). Forward and reverse sequences were generated for each DNA sample. Sequence trace alignment and polymorphism detection was carried out using recent versions of Phred/Phrap (Ewing et al. 1998. Ewing and Green 1998) and Consed (Gordon et al. 1998).

SNPs discovered through the above described sequencing efforts were analyzed for genotypes matching that expected if the SNPs are associated with/causal to the horned/polled phenotype. The expected genotypic profile is: a) all sires homozygous for one allele, and all sons either homozygous for the other allele (or possibly heterozygous). Those SNPs listed in Table 1 showed 100% concordance with the predicted genotypic profile, and thus showed association with the horned/polled phenotype.

### Example 6

### Development of an Improved Genetic Profile

The 32 markers selected for inclusion in the marker set were derived from analyses of the results of genome scan experiments described in Example 1. The descriptor “anchor marker” was used to define those markers that represented the marker within a QTL region that best represented that QTL region in comparison to other markers that also exist within the QTL region. Anchor markers were identified by first selecting those markers that had an observed—maximum F statistic>1. If multiple markers around a locus fit this criterion then the marker with the largest observed—maximum F statistic was selected as an anchor, with the further stipulation that no other anchor markers could exist within 5 centimorgans of the new anchor marker.

The list of 62 anchor markers was then sorted by allele frequency to screen out markers with minor allele frequencies less than 30% (the rationale behind this screening was to remove those markers that may have a biased effect estimate due to low sample number). The resulting 32 markers are included in Table 2 below.

Based on these results, animals having the preferred allele described in table 2 are expected to have improved genetic and phenotypic characteristics, including fitness and productivity traits. Animals having the preferred allele described in Table 2 in combination with the alleles associated with the polled phenotype as described in Table 1 are expected to have particularly valuable genetic and phenotypic characteristics including fitness, productivity, and polled traits.

### Example 7

### Determination of a Genetic Profile of a Bull

A sample of genetic material can be obtained from any biological source containing representative DNA, but preferred methods typically employ the use of blood, semen, hair, or saliva. Once the sample has been obtained, standard methods of genotyping are used to determine the alleles of the sample at markers listed in Tables 1 and 2. A sample having more alleles found in the “preferred” column of Table 2 would indicate superior genetic and/or phenotypic performance in comparison with a sample having fewer alleles found in the “preferred” column. If the genetic profile of the bull is homozygous for at least 12 alleles in the preferred orientation described in Table 2, it is selected for breeding purposes.

### Example 8

### Determination of a Genetic Profile of a Bovine Product

A representative sample of the product comprising DNA is extracted from the product. For example, when testing milk or dairy products, DNA may be retrieved from the leucocytes cells contained therein. When testing bovine meat products. DNA can be extracted from the muscle fibers. For samples comprising large concentrations of cells and/or DNA, standard methods of genotyping are used to determine the alleles of the sample at markers listed in Tables 1 and 2. Preferably when evaluating the genetic profile of bovine products, DNA from at least about 50 individual cells are used to determine the genetic profile. However, recent advances in the field of DNA extraction and replication allow for determining genetic content from a sample as small as one cell.

### Example 9

### Determination of a Genetic Profile of Bull Semen

Even though semen contains haploid cells, these cells can still be used to create a genetic profile by genotyping a large number of cells. The first step is to get a semen straw or sample that contains sufficiently large number of sperm cells (e.g., >1,000,000 cells). The second step is to extract DNA from the semen straw (namely a pool of a large number of sperm cells). The extracted DNA is then to be used to genotype markers listed in Table 1 and the Sequence Listing. These genotype results will include information on both strands of DNA of the parent animal. Therefore, the genotype data can be used for determination of the genetic profile as described above.

### REFERENCES

The references cited in this application, both above and below, are specifically incorporated herein by reference.

Non-Patent Literature
Abdel-Azim, G and Freeman, A E, (2002) J. Dairy Sci, 85:1869-1880.
Blott, S., Kim, J. J., Moisio, S., et al. (2003). Genetics 163: 253-266.
Brenneman R A, Davis S K, Sander J O, Bums B M, Wheeler T C, Turner J W, Taylor J F (1996). The polled locus maps to BTA1 in a Bos indicus×Bos taurus cross. Journal of Heredity 87:156-161.
Ciobanu, D C, Bastiaansen, J W M, Longergan, S M, Thomsen, Dekkers, J C M, Plastow, G S, and Rothschild, M F, (2004) J. Anim. Sci. 82:2829-39.
Cohen-Zinder, M. et al. (2005) Genome Res. 15:936-44.
Davis, G P and DeNise, S K, (1998) J. Anim. Sci. 76:2331-39.
Dekkers, J C M, and Chakraborty, R. (2001) J. Anim. Sci. 79:2975-90.
Demars J. Riquet J, Feve K. Gautier M. Morisson M, Demeure O, Renard C, Chardon P, Milan D. (2006), BMC Genotnics, 24:7-13
Drögemüller C, Wöhlke A, Momke S. Distl O (2005). Fine mapping of the polled locus to a 1-Mb region on bovine chromosome 1q12. Mammalian Genome 16:613-620.
Du and Hoeschele, (2000) Genetics 156:2051-62.
Ducrocq, V. 1987. An analysis of length of productive life in dairy cattle. Ph.D. Diss. Cornell Univ., Ithaca, N.Y.: Univ. Microfilms Int., Ann Arbor, Mich.
Everts-van der Wind A. Larkin D M, Green C A, Elliott J S, Olmstead C A, Chiu R, Schein J E, Marra M A, Womack J E, Lewin H A. (2005) Proc Nutl Acud Sci USA, 20:102(51):18526-31.
Falconer, D S, and Mackay, T F C, (1996) Introduction to Quantitative Genetics. Harlow, UK: Longman.
Fernando, R, and Grossman, M, (1989) Marker assisted selection using best linear unbiased prediction. Genetics Selection Evolution 21:467-77.
Franco, M M, Antunes, R C, Silva, H D, and Goulart, L R (2005) J. Appl. Genet. 46(2):195-200.
Georges M. Drinkwater R, King T. Mishra A. Moore S S, Nielsen D. Sargeant L S, Sorensen A, Steele M R, Zhao X, Womack J E, Hetzel J (1993). Microsatellite mapping of a gene affecting horn development in Bos taurus. Nature Genetics 4:206-210.
Grisart, B. et al. (2002) Genome Res. 12:222-231
Grosz, M D, Womack, J E, and Skow. L C (1992) Genomics, 14(4):863-868.
Harlizius B. Tammen I, Eichler K. Eggen A, Hetzel D J (1997). New markers on bovine chromosome 1 are closely linked to the polled gene in Simmental and Pinzgauer cattle. Mammalian Genome 8:255-257.
Hayes, B. and Goddard, M E, (2001) Genet. Sel. Evol. 33:209-229.
Hayes, B, Chamberlain, A. J., Goddard, M. E. (2006) Proc. 8^{th} WCGALP 22:(16).
Kaminski, S. Ahman, A. Rusc, A, Wojcik, E. and Malewski, T (2005) J. Appl. Genet. 46(1):45-58.
Kuhn, C. et al. (2004). Genetics 167:1873-81.
Kwok P Y, Methods for genotyping single nucleotide polymorphisms, (2001), Annu. Rev. Genomics Hum. Genet., 2:235-258.
Long C R, Gregory K E (1978). Inheritance of the horned scuffed, and polled condition in cattle. Journal of Heredity 69:395 400.
Meuwissen, T H E, and Van Arendonk. JAM, (1992) J. Dairy Sci. 75:1651-1659.
Meuwissen, T H E, Hayes, B J, and Goddard, M E, (2001) Genetics. 157:1819-29.
Rothschild and Plastow, (1999), AgBioTechNet 10:1-8.
Schaeffer, L R (2006) J. Anim. Breed. Genet. 123:218-223.
Schmutz S M, Marquess F L S, Berryere T G, Moker J S (1995). DNA marker assisted selection of the polled condition in Charolais cattle. Mammalian Genome 6:710-713.
Schnabel, R. et al. (2005) PNAS 102:6896-6901.
Schrooten, C, Bovenhuis, H, van Arendonk, J A M, and Bijma, P (2005) J. Dairy Sci. 88:1569-1581.
Sharma, B S, Jansen, G B. Karrow, N A, Kelton, D, and Jiang, Z, (2006) J. Dairy Sci. 89:3653-3663.
Short, T H, et al. (1997) J. Anim. Sci. 75:3138-3142.
Spelman, R J and Bovenhuis, H, (1998) Animal Genetics, 29:77-84.
Spelman, R J and Garrick, D J, (1998) J. Dairy Sci, 81:2942-2950.
Stearns, T M, Beever, J E, Southey, B R, Ellis, M, McKeith, F K and Rodriguez-Zas, S L, (2005) J. Anim. Sci. 83:1481-93.
Syvänen A C, Accessing genetic variation: genotyping single nucleotide polymorphisms, (2001) Nat. Rev. Genet. 2:930-942.
VanRaden, P. M. and E. J. H. Klaaskate. 1993. J. Dairy Sci. 76:2758-2764.
Verrier, E, (2001) Genet. Sel. Evol. 33:17-38.
Villanueva, B, Pong-Wong, R. Fernandez, J, and Toro. M A (2005) J. Anim. Sci. 83:1747-52.
White W T, Ibsen H L (1936). Horn inheritance in Galloway-Holstein cattle crosses. Journal of Genetics 32:33-49.
Williams, J L, (2005), Rev. Sci. Tech. Off. Int. Epiz. 24(1):379-391.
Windig, J J, and Meuwissen, T H E, (2004) J. Anim. Breed. Genet. 121:26-39.
Winter, A. et al. (2002). PNAS, 99:9300-9305.
Womack, J. (1987), Dev. Genet. 8(4):281-293.
Wunderlich K R, Abbey C A, Clayton D R, Song Y, Schein J E, Georges M, Coppieters W, Adelson D L, Taylor J F, Davis S L, Gill C A (2006). A 2.5-Mb contig constructed from Angus, Longhorn and horned Hereford DNA spanning the polled interval on bovine chromosome 1. Animal Genetics 37:592-594.
Yasue H, Kiuchi S. Hiraiwa H, Ozawa A, Hayashi T, (2006), Cytogenet. Genome Res., 112(1-2):121-125.
Youngerman, S M, Saxton, A M, Oliver, S P, and Pighetti, G M, (2004) J. Dairy Sci. 87:2442-2448.

Patent Literature

| Patent/Publication | | | |
|---|---|---|---|
| Number | Title | Inventors | Pub. Date |
| U.S. Pat. No. 5,041,371 | Genetic marker for | Cowan, Charles M.; | Aug. 20, 1991 |
| | superior milk products in | Dentine, Margaret R.; | |
| | dairy cattle | Ax, Roy L.; Schuler, | |
| | | Linda A. | |
| U.S. Pat. No. 5,374,523 | Allelic variants of bovine | Collier, Robert J.; | Dec. 20, 1994 |
| | somatotropin gene:genetic | Hauser, Scott D.; Krivi, | |
| | marker for superior milk | Gwen G.; Lucy, | |
| | production in bovine | Matthew C. | |
| U.S. Pat. No. 5,582,987 | Methods for testing bovine | Lewin, Harris A.; van | Dec. 10, 1996 |
| | for resistance or | Eijk, Michiel J. T. | |
| | susceptibility to persistent | | |
| | lymphocytosis by detecting | | |
| | polymorphism in bola-dr3 | | |
| | exon 2 | | |
| U.S. Pat. No. 5,614,364 | Genetic marker for | Tuggle, Christopher K.; | Mar. 25, 1997 |
| | improved milk production | Freeman, Albert E. | |
| | traits in cattle | | |
| US2003039737 | Population of dairy cows | Cooper, Garth J. S. | Feb. 27, 2003 |
| A1 | producing milk with | | |
| | desirable characteristics | | |
| | and methods of making | | |
| | and using same | | |
| US2004076977 | Marker assisted selection | Georges, Michel | Apr. 22, 2004 |
| A1 | of bovine for improved | Alphonse Julien; | |
| | milk production using | Coppieters, Wonter | |
| | diacylglycerol | Herman Robert; Grisart, | |
| | acyltransferase gene dgat1 | Bernard Marie-Josee Jean; | |
| | | Shell, Russell Grant; Jean | |
| | | Reid, Suzanne; Ford, | |
| | | Christine Ann; Spelman, | |
| | | Richard John | |
| US2004234986 | Method of testing a | Fries, Hans-Rudolf; | Nov. 25, 2004 |
| A1 | mammal for its | Winter, Andreas | |
| | predisposition for fat | | |
| | content of milk and/or its | | |
| | predisposition for meat | | |
| | marbling | | |
| US2004241723 | Systems and methods for | Marquess, Foley Leigh | Dec. 2, 2004 |
| A1 | improving protein and milk | Shaw; Laarveld, Bernard; | |
| | production of dairy herds | Cleverly Buchanan, | |
| | | Fiona; Van Kessel, | |
| | | Andrew Gerald; Schmutz, | |
| | | Sheila Marie; Waldner, | |
| | | Cheryl; Christensen, | |
| | | David | |
| US2004254104 | Marker assisted selection | Blott, Sarah; Kim, Jong- | Dec. 16, 2004 |
| A1 | of bovine for improved | Joo; Schmidt-Kuntzel, | |
| | milk composition | Anne; Cornet, Anne; | |
| | | Berzi, Paulette; | |
| | | Cambisano, Nadine; | |
| | | Grisart, Bernard; Karim, | |
| | | Latifa; Simon, Patricia; | |
| | | Georges, Michel; Farnir, | |
| | | Frederic; Coppieters, | |
| | | Wouter; Moisio, Sirja; | |
| | | Vilkki, Johanna; | |
| | | Spelman, Richard; | |
| | | Johnson, Dave; Ford, | |
| | | Christine; Snell, Russell | |
| US2005123929 | Methods and compositions | Khatib, Hasan | Jun. 9, 2005 |
| A1 | for genetically detecting | | |
| | improved milk production | | |
| | traits in cattle | | |
| US2005136440 | Method for identifying | Renaville, Robert; | Jun. 23, 2005 |
| A1 | animals for milk | Gengler, Nicolas | |
| | production qualities by | | |
| | analysing the | | |
| | polymorphism of the pit-1 | | |
| | and kappa-casein genes | | |
| US2005137805 | Gene expression profiles | Lewin, Harris A.; Liu, | Jun. 23, 2005 |
| A1 | that identify genetically | Zonglin; Rodriguez-Zas, | |
| | elite ungulate mammals | Sandra; Everts, Robin E. | |
| US2005153317 | Methods and systems for | DeNise, Sue; Rosenfeld, | Jul. 14, 2005 |
| A1 | inferring traits to breed and | David; Kerr, Richard; | |
| | manage non-beef livestock | Bates, Stephen; Holm, | |
| | | Tom | |
| US2005053328 | Method and Markers for | DeNise et al | Jul. 14, 2005 |
| A1 | Determining the Genotype | | |
| | of Horned/Polled Cattle | | |
| US2006037090 | Selecting animals for | Andersson, Leif; | Feb. 16, 2006 |
| A1 | desired genotypic or | Andersson, Goran; | |
| | potential phenotypic | Georges, Michel; Buys, | |
| | properties | Nadine | |
| US2006094011 | Method for altering fatty | Morris, Christopher | May 4, 2006 |
| A1 | acid composition of milk | Anthony; Tate, Michael | |
| | | Lewis | |
| US2006121472 | Method for determining the | Prinzenberg, Eva-Maria; | Jun. 8, 2006 |
| A1 | allelic state of the 5′-end of | Erhardt, George | |
| | the $g(a)s1-casein gene | | |
| US2006166244 | Dna markers for increased | Schnabel, Robert D.; | Jul. 27, 2006 |
| A1 | milk production in cattle | Sonstegard, Tad S.; Van | |
| | | Tassell, Curtis P.; | |
| | | Ashwell, Melissa S.; | |
| | | Taylor, Jeremy F. | |
| US2007134701 | Method and Markers for | DeNise et al | Jun. 14, 2007 |
| A1 | Determining the Genotype | | |
| | of Horned/Polled Cattle | | |

### Description of the Tables

Table 1: provides the SEQ ID numbers of the SNPs associated with the polled/homed phenotype as described herein and the allele of each SNP as it corresponds to polled or horned.

Table 2: provides the SEQ ID numbers of SNPs useful in constructing genetic profiles with respect to economically significant traits such as productivity and fitness traits.

TABLE 1
SNPs associated with the polled trait.

| SEQ | Polled | Horned |
|---|---|---|
| ID | Allele | Allele |
| 1 | C | T |
| 2 | T | C |
| 3 | T | C |
| 4 | G | A |
| 5 | T | C |
| 6 | A | G |
| 7 | T | C |
| 8 | C | T |
| 9 | G | A |
| 10 | A | G |
| 11 | G | A |
| 12 | A | C |
| 13 | C | T |

TABLE 2
SNPs associated with traits of economic significance.
In each case, the preferred allele is listed as “Allele 1”

| SEQ | | Preferred | |
|---|---|---|---|
| ID | SNP_ID | Allele | Allele 2 |
| 15 | NBYA_342584 | G | A |
| 16 | NBYA_342716 | T | C |
| 17 | NBYA_342832 | T | G |
| 18 | NBYA_343138 | G | T |
| 19 | NBYA_343185 | A | G |
| 20 | NBYA_343186 | A | G |
| 21 | NBYA_343307 | T | C |
| 22 | NBYA_343573 | T | C |
| 23 | NBYA_343580 | G | A |
| 24 | NBYA_345616 | T | C |
| 25 | NBYA_345686 | T | C |
| 26 | NBYA_346236 | T | C |
| 27 | NBYA_347875 | G | T |
| 28 | NBYA_348097 | C | A |
| 29 | NBYA_348346 | T | C |
| 30 | NBYA_349346 | A | G |
| 31 | NBYA_349348 | A | G |
| 32 | NBYA_349455 | G | A |
| 33 | NBYA_350413 | A | C |
| 34 | NBYA_350479 | G | C |
| 35 | NBYA_350505 | A | G |
| 36 | NBYA_350590 | T | C |
| 37 | NBYA_350805 | G | C |
| 38 | NBYA_350936 | G | A |
| 39 | NBYA_352717 | G | A |
| 40 | NBYA_353064 | A | G |
| 41 | NBYA_353076 | A | G |
| 42 | NBYA_353135 | T | C |
| 43 | NBYA_353365 | G | A |
| 44 | NBYA_353833 | C | T |
| 45 | NBYA_353947 | T | A |
| 46 | NBYA_354471 | T | A |

1. A method for allocating a bovine animal for use according to the animal's genetic profile, the method comprising:
a. determining the animal's genotype at 12 or more loci, wherein each locus contains a single nucleotide polymorphism (SNP) having at least two allelic variants; and wherein m herein at least 12 SNPs are selected fro the SNPs described in Table 2 and the Sequence Listing;
b. analyzing the determined genotype of the at least one evaluated animal; and
c. allocating the animal or use based on it's determined genetic profile;
wherein said animal is homozygous for the preferred allele for at least 12 SNPs selected from the SNPs described in Table 2 and the Sequence Listing.

2. The method of claim 1 wherein part “a.” further comprises determining the animal's genotype at one or more additional loci that each contain(s) at least one additional SNP having at least two allelic variants; wherein at least one of the additional SNP(s) is/are associated with the polled trait and is/are selected from the SNPs described in Table 1 and the sequence listing; and wherein said animal is heterozygous for one or more of these additional SNPs.

3. The method of claim 1 wherein part “a.” further comprises determining the animal's genotype at one or more additional loci that each contain(s) at least one additional SNP having at least two allelic variants; wherein at least one of the additional SNP(s) is/are associated with the polled trait and is/are selected from the SNPs described in Table 1 and the sequence listing; and wherein said animal is homozygous for one or more of these additional SN Ps.

4. The method of claim 1 wherein said animal is homozygous for the preferred allele at each of at least 13 SNPs selected from the SNPs described in Table 2 and the sequence listing.

5. The method of claim 1 wherein said animal is homozygous for the preferred allele at each of at least 14 SNPs selected from the SNPs described in Table 2 and the sequence listing.

6. The method of claim 1 wherein said animal is homozygous for the preferred allele at each of at least 15 SNPs selected from the SNPs described in Table 2 and the sequence listing.

7. The method of claim 1 wherein said animal is homozygous for the preferred allele at each of at least 16 SNPs selected from the SNPs described in Table 2 and the sequence listing.

8. The method of claim 1 wherein said animal is homozygous for the preferred allele at each of at least 18 SNPs selected from the SNPs described in Table 2 and the sequence listing.

9. The method of claim 1 wherein said animal is homozygous for the preferred allele at each of at least 20 SNPs selected from the SNPs described in Table 2 and the sequence listing.

10. The method of claim 1 wherein said animal is polled.

11. A method for allocating a potential parent bovine animal for use according to the animal's genetic profile, the method comprising:
a. determining the animal's genotype at 12 or more loci, wherein each locus contains a single nucleotide polymorphism (SNP) having at least two allelic variants; and wherein at least 12 SNPs are selected from the SNPs described in Table 2 and the Sequence Listing;
b. analyzing the determined genotype of the at least one evaluated animal; and
c. allocating at least one animal for breeding use based on it's genotype;
wherein said animal is homozygous for the preferred allele for at least 12 SNPs selected from the SNPs described in Table 2 and the sequence listing.

12. The method of claim 11 wherein part “a.” further comprises determining the animal's genotype at one or more additional loci that each contain(s) at least one additional SNP having at least two allelic variants; wherein at least one of the additional SNP(s) is/are associated with the polled trait and is/are selected from the SNPs described in Table 1 and the sequence listing; and wherein said animal is heterozygous for one or more of these additional SNPs.

13. The method of claim 11 wherein part “a.” further comprises determining the animal's genotype at one or more additional loci that each contain(s) at least one additional SNP having at least two allelic variants; wherein at least on of the additional SNP(s) is/are associated with the polled trait and is/are selected from the SNPs described in Table 1 and the sequence listing; and wherein said animal is homozygous for the one or more of these additional SNPs.

14. The method of claim 11 wherein said animal is homozygous for the preferred allele at each of at least 13 SNPs selected from the SNPs described in Table 2 and the sequence listing.

15. The method of claim 11 wherein said animal is homozygous for the preferred allele at each of at least 14 SNPs selected from the SNPs described in Table 2 and the sequence listing.

16. The method of claim 11 wherein said animal is homozygous for the preferred allele at each of at least 15 SNPs selected from the SNPs described in Table 2 and the sequence listing.

17. The method of claim 11 wherein said animal is homozygous for the preferred allele at each of at least 16 SNPs selected from the SNPs described in Table 2 and the sequence listing.

18. The method of claim 11 wherein said animal is homozygous for the preferred allele at each of at least 18 SNPs selected from the SNPs described in Table 2 and the sequence listing.

19. The method of claim 11 wherein said animal is homozygous for the preferred allele at each of at least 20 SNPs selected from the SNPs described in Table 2 and the sequence listing.

20. The method of claim 11 wherein said potential parent bovine animal is polled.

21. A method of producing progeny from bovine animals comprising:
a) identifying at least one potential parent animal that has been allocated for breeding in accordance with the method of claim 1;
b) producing progeny from the allocated animal through a process selected from the group consisting of:
i) natural breeding;
ii) artificial insemination;
iii) in vitro fertilization; and
iv) collecting sennen/spermatozoa or at least one ovum from the animal and contacting it, respectively, with ovum/ova or semen/spermatozoa from a second animal to produce a conceptus by any means.

22-46. (canceled)
